(19) United States
(12) Patent Application Publication
(10) Pub. No.: US 2025/0043276 A1
(43) Pub. Date: Feb. 6, 2025

(54) Targeted Enrichment of Nucleic Acid Sequences

(71) Applicant: University of Oregon, Eugene, OR (US)

(72) Inventors: Calin Plesa, Eugene, OR (US); Netanya K Villegas, Eugene, OR (US)

(21) Appl. No.: 18/239,043

(22) Filed: Aug. 28, 2023

Related U.S. Application Data
(60) Provisional application No. 63/401,072, filed on Aug. 25, 2022.
(60) Provisional application No. 63/401,127, filed on Aug. 26, 2022.

(51) Int. Cl. C12N 15/10

(52) U.S. Cl. CPC C12N 15/1093

(57) ABSTRACT

Compositions and methods for enriching pools of nucleic acid sequences, particularly for using single guide RNAs and deactivated Cas proteins to selectively enrich pools of nucleic acid molecules for predetermined sequences, such as perfect gene assemblies and methods of making and using single guide RNAs are provided.

### RELATED APPLICATIONS

This application claims priority to U.S. Provisional Application No. 63/401,072, filed on Aug. 25, 2022 and U.S. Provisional Application No. 63/401,127, filed on Aug. 26, 2022, both which are hereby incorporated by reference in their entirety.

### STATEMENT OF GOVERNMENT INTERESTS

This invention was made with government support under 2032259 awarded by the National Science Foundation. The government has certain rights in the invention.

### FIELD OF DISCLOSURE

This disclosure relates to methods of enriching pools of nucleic acid sequences, particularly to the use of single guide RNAs and inactive Cas proteins to selectively enrich pools of nucleic acid molecules for predetermined sequences and methods of making and using single guide RNAs.

### SEQUENCE LISTING INCORPORATION BY REFERENCE

The Sequence Listing is submitted as an XML file in the form of the file named “1505-110875-02_Sequence_Listing.xml” (41,162 bytes), which was created on Jun. 5, 2024, which is incorporated by reference herein.

### BACKGROUND OF THE DISCLOSURE

All gene assembly (and oligonucleotide synthesis) approaches produce a mixed population containing both perfect and imperfect assemblies (correct and incorrect DNA sequence). Strategies to select or enrich perfect gene assemblies can be broadly classified into three groups: functional selection, enzymatic error correction, and targeted enrichment:

Functional selection is limited to protein coding sequences and involves an in-vivo selection which removes loss-of-function variants. Since deletions comprise the majority of errors and most of these lead to frameshifts, creating in-frame fusions of the protein of interest to a selectable marker allows for the selection of assemblies without frameshifts. A complicating factor in this approach is translation (re) initiation at both downstream ATGs and non-AUG start codons which can bypass frameshift errors and reduce the effectiveness of the selection.

Enzymatic error correction typically involves melting and reannealing gene assemblies to form heteroduplexes (one perfect strand hybridized to an error containing strand) where the mismatches between the two strands can be cleaved (Endonuclease V, CorrectASE, etc.) or bound and removed (MutS). This approach is a good way to individually isolate short constructs but is difficult to implement for gene libraries without isolation due to cross-hybridization effects. Moreover, enzymatic correction often fails for long constructs where nearly all molecules have some errors.

Targeted enrichment of specific molecules (e.g., perfect assemblies or sub-libraries) in a sequence population can involve physically isolating perfect molecules from a cluster on a sequencing flow cell or methods which use barcode-tagged molecules. The former methods require highly specialized equipment and are not compatible with many current next generation sequencing (“NGS”) machines. In the latter approaches, often termed dial-out PCR, molecules are tagged with a random barcode, sequenced and barcodes corresponding to sequences of interest are identified. Barcoded primers are used to PCR amplify the perfect assemblies or sub-libraries out of the pooled library. See, e.g., Schwartz J J, Lee C, Shendure J. 2012. Accurate gene synthesis with tag-directed retrieval of sequence-verified DNA Molecules. Nat. Methods 9:913-15. DOI: 10.1038/nmeth.2137 which is hereby incorporated by reference int its entirety.

This approach does not scale well as each construct requires its own amplification reaction and any approach which isolates individual members from a pooled library increases overall costs substantially.

There remains a need for a low-cost multiplexed tag-directed retrieval approach which will maintain the enriched library in a pooled format while remaining cost-effective at large scale.

### SUMMARY

In one embodiment of the invention, a method of enriching a predetermined nucleic acid molecule in a starting set of nucleic acid sequences comprising the steps of: providing a starting set of nucleic acid sequences, the starting set comprising a plurality of nucleic acid sequences each of which comprises a unique subsequence, contacting the starting nucleic acid sequence set with a nucleic acid targeting system that specifically binds to the unique subsequence of the of predetermined nucleic acid molecule, separating nucleic acid targeting system from the starting nucleic acid sequence set, and releasing the predetermined nucleic acid molecule from the nucleic acid targeting system such that a second nucleic acid molecule set if formed in which the predetermined nucleic acid molecule is enriched as compared to the starting nucleic acid set is provided. In one embodiment, the predetermined nucleic acid molecule is a DNA molecule, the starting nucleic acid sequence set is a DNA sequence set and the nucleic acid targeting system is an RNA guided targeting system. In particular embodiments, the RNA guided targeting system is a Cas9, Cas12, Cas13a, Cas13b, Cas12f, Cascade-Cas3, prokaryotic argonautes (*Marinitoga piezophila* (MpAgo), *Thermotoga profunda* (TpAgo), or *Rhodobacter sphaeroides* (RsAgo)) system.

In one embodiment, the RNA guided system is a CRISPR Cas9 system comprising a Cas9 nuclease and the sequences in the starting DNA sequence set comprises a protospacer adjacent motif, particularly the s 5′-NGG-3′. In one embodiment, the Cas9 nuclease is deactivated.

In one embodiment, the plurality of nucleic acid sequence in the starting nucleic acid set of any of the above embodiments, comprises at least 10², 10³, 10⁴, 10⁵, 10⁶, 10⁷, or 10⁸ nucleic acid sequences to about 10, 20, 30, 40 or 50×10⁹ sequences each of which comprise a unique random sequence. In one embodiment, the starting nucleic acid sequence set of any of the above embodiments comprises a plurality of predetermined nucleic acid molecule each comprising a size, wherein the size of the predetermined nucleic acid molecule is at least 100, 200, 300, 400, 500, 1000, 2000, 3000, 4000 or 5000 to about 10⁶ nucleotides.

In a particular embodiment, the plurality of the predetermined nucleic acid molecules of any of the above embodiments comprises a plurality of sizes, wherein the plurality of sizes is in the range of 10 to 5000, 50 to 4000, 100 to 3000, 500 to 3000 or 500 to 2000 nucleotides.

In one embodiment of this aspect of the invention, the starting nucleic acid set comprises at least 100, 150, 200, 300, 400 or 500 ng of DNA where the enrichment reaction is run in a final volume of about 30 uls. In further embodiments of the invention, the second nucleic acid sequence set is treated with proteinase K prior to quantification. In a further embodiment, the enrichment reaction is run for about 15 minutes to no more than 30, 45 or 60 minutes. In further embodiments, the second nuclei acid sequence set of any of the above embodiments is washed at least 6, 7, 8, or 9 times with a total wash volume of at least 2, 3, 4, or 5 mls.

In some embodiments of the invention, a quantity of the predetermined nucleic acid molecule in the second nucleic acid set is enriched by at least one or two orders of magnitude as compared to a quantity of the predetermined nucleic acid molecule in the starting nucleic acid set. In one embodiment of this aspect of the invention, at least 30, 40, 50, 60, 70, 80, or 90% of the nucleic sequences in the second nucleic acid molecule set are the plurality of predetermined sequences. In one embodiment of this aspect of the invention, at least 40%, 50%, 60% 70%, 80% or 90% of each predetermined sequence is perfect.

In one aspect of the invention, a method of preparing a library of single guide RNA molecules, comprising: providing a plurality of double stranded DNA oligonucleotide molecules wherein each oligonucleotide molecule comprises a set of 2 orthogonal primer sequences, a T7 promoter, a spacer sequence, a scaffold overhang sequence, a type 2 restriction site and a stop codon, providing a plurality of double stranded scaffold fragment sequences having a 5′ end, incubating the plurality of oligonucleotide molecules and scaffold fragment sequences with a type II restriction enzyme and a ligase in the same reaction mixture, wherein the type 2 restriction enzyme creates a 5′ overhang on the spacer oligonucleotide and on the scaffold oligonucleotide wherein the 5′ overhang on the spacer oligonucleotide is complementary to the 5′ overhang on the scaffold oligonucleotide thereby providing a library of assembled single guide RNA DNA template molecules, and transcribing the single guide RNA DNA template molecules into a plurality of single guide RNA molecules.

In some embodiments the type 2 restriction enzyme is AcuI, AlwI, BaeI, BbsI, BbsI-H, BbvI, BccI, BceAI, BcgI, BciVI, BcoDI, BfuAI, Bmr, BpmI, BpuEI, BsaI-HF®v2, BsaXI, BseRI, BsgI, BsmA, BsmBI-v2, BsmFI, BsmI, BspCNI, spMI, BspQI, BsrD, BsrI, BtgZI, BtsCI, BtsI-v2, BtsIMutI, CspC, EarI, EciI, Esp3I, FauI, FokI, Hga,I, HphI, pyAV, MboII, MlyI, MmeI, MnlI, NmeAIII, PaqCI, PleI, SapI or SfaNI restriction enzyme.

In some embodiments, the double stranded DNA oligonucleotide molecules are prepared from a single stranded DNA oligonucleotide template by primer extension while in some embodiments double stranded DNA oligonucleotide molecules are prepared from a single stranded DNA oligonucleotide template by PCR amplification. In some embodiments the spacer sequence for use in the methods of the invention comprises, 5 to 100, 10 to 90, 12 to 80, 15 to 70, 16 to 60, 17 to 50, 18 to 40, 19 to 30, 26 to 72, 19 to 21 or 20 nucleotides. In some embodiments of this aspect of the invention the spacer sequence does not comprise protospacer adjacent motif or type 2 restriction site.

22. In some embodiments the ratio of the plurality of scaffold fragment sequences to the plurality of oligonucleotide sequences during sgRNA assembly of 2 to 1. In some embodiments, the spacer sequences of the plurality of oligonucleotide molecules target more than 2, 20, 25, 50, 60, 70, 80, 90, 100, 10, 10², 10³, 10⁴, 10⁵, 10⁶, 10⁸ to about 10⁹ different nucleic acid molecules. And in one embodiment, an sgRNA library produced by any of the above methods of the invention is provided.

### BRIEF DESCRIPTION OF THE DRAWINGS

FIG. 1 is a schematic depicting a method of enriching a population of nucleic acid molecules for perfect assemblies using barcode directed deactivated Cas9.

FIG. 2 is a schematic depicting a method of enriching a population of nucleic acid molecules for perfect assemblies where they are cyclized and are selected for while the incorrect assemblies remain linear.

FIG. 3 is a gel showing targeted enriched nucleic acid sequences of about 600 base pairs after being enriched.

FIG. 4a is a graph showing the Gini coefficient and Lorenz curve of the mCherry RFP library prior to enrichment while FIG. 4b is a graph showing the Gini coefficient and Lorenz curve for the mCherry RFP library after enrichment.

FIG. 5a is a graph showing the rank ordered number of sequencing reads for each observed barcode (vertical lines) in the unenriched target nucleic acid molecule population. While FIG. 5b is a graph showing the rank ordered number of sequencings reads for each observed barcode after enrichment. The vertical lines show the targeted 18 selected barcodes after enrichment.

FIG. 6a is a graph showing the log 2 enrichment values after targeted enrichment. The vertical spacers show 18 targeted barcodes. While FIG. 6b shows the same data as FIG. 6a except low read number barcodes have been removed for clarity.

FIG. 7a is a graph that plots the correlation in the population fractions between barcodes in the unenriched library (x-axis) and the barcodes in the enriched population (y-axis). FIG. 7b is the same plot as FIG. 7a except that the plot has been separated into enriched and unenriched populations.

FIG. 8 is a graph showing the amount of Log 2 enrichment observed for the enriched population after washes (y-axis) relative to the number the fraction of the corresponding original population (x-axis).

FIG. 9a is an enrichment violin plot showing the difference between the Log 2 enrichment after washes (vertical axis) for off target and target nucleic acid sequence populations (horizontal axis).

FIG. 9b is a violin plot showing the difference between Log 2 enrichment of targeted populations (vertical axis) for different bead washing conditions (horizontal axis).

FIG. 10 is a graph showing the total observed population fraction enrichment factor (vertical axis) as a function of the total cumulative wash volume used to wash the bound beads (horizontal axis).

FIG. 11 is a schematic illustrating a multiplexed sgRNA library production method of the invention and an illustrative spacer oligonucleotide template to be used in the sgRNA synthesis methods of the invention.

FIG. 12 is a photograph of two gels, an agarose E-gel (2%) of full length sgRNA oligonucleotides, 121 bp, for an assembled individual sgRNA and a pool of 18 assembled sgRNA oligonucleotides (left side) and a denaturing TBE-Urea gel 10% of individually transcribed sgRNA 1 and a pool of 18 transcribed sgRNA oligonucleotides right side.

FIG. 13 is a Lorenz curve plotting the cumulative share of reads on the vertical axis versus the % of sgRNA spacer by fraction of total read, where the Gini Coefficient of 0.97.

FIG. 14 is a graph, plotting sgRNA reads on the vertical axis versus GC content in percentage on the horizontal axis.

FIG. 15 is a violin plot of Log 2 Fold Enrichment values (vertical axis) for the indicated conditions and controls (horizontal axis).

FIG. 16 is a boxplot showing the Log 2fold enrichment (horizontal axis) for the off-target controls and conditions indicated on the vertical axis.

FIG. 17 is a graph that plots the Gina coefficient (vertical axis) by library (horizontal axis).

FIG. 18 is a graph that plots the fraction of spacer sequences of observed (vertical axis) by library (horizontal axis).

FIG. 19 is a graph that plots the sequencing depth (vertical axis) by library (horizontal axis) in the sequencing runs that resulted in the data of FIG. 17 and FIG. 18.

FIG. 20 is a graph that plots the total number of spacer oligo sequence reads to mutated spacer reads (horizontal axis) by library.

### DETAILED DESCRIPTION

Aspects of the present invention are directed to selecting, in multiplex, certain nucleic acid molecules containing desirable sequences out of large populations of mixed nucleic acid molecules containing many different sequences. In some aspects of the invention, this includes enriching DNA molecules having perfect sequences out of a mixed population of molecules with both perfect sequences and sequences with errors. In some aspects of the invention, it also includes taking large libraries or population of DNA molecules and selecting subset libraries of interest.

Aspects of the present invention are also directed to making and using single guide RNAs (“sgRNAs”), particularly making in sgRNA libraries that target many sequences of interest. In some aspects of the invention, all steps of the manufacture of single guide RNA libraries are conducted in vitro.

While the terminology used in the instant disclosure is standard within the art, definitions of certain terms are provided herein to assure clarity and definiteness to the meaning of the claims. Units, prefixes, and symbols can be denoted in their SI accepted form. As described herein, any concentration range, percentage range, ratio range or integer range is to be understood to include the value of any integer within the recited range and, when appropriate, fractions thereof (such as one-tenth and one-hundredth of an integer), unless otherwise indicated. The section headings used herein are for organizational purposes only and are not to be construed as limiting the subject matter described.

As used in the instant disclosure, except as otherwise expressly provided herein, each of the following terms shall have the meaning set forth below. Additional definitions are set forth throughout the disclosure.

Unless otherwise noted, the terms “a” or “an” are to be construed as meaning “at least one or more of”.

The term “about” as used in connection with a numerical value throughout the specification and the claims denotes an interval of accuracy, familiar and acceptable to a person skilled in the art. In general, such interval of accuracy is plus or minus. 15%.

The term “annealing” as used herein refers to the process of heating and cooling two single-stranded oligonucleotides with complementary sequences resulting in hydrogen bonds forming between the two sequences.

As used herein the term protospacer adjacent motif (hereinafter “PAM”) is a 3-4 base pair sequence that is 3 to 4 bases downstream of the Cas9 nuclease cut site on the targeted DNA sequence. As used herein the term “seed” sequence refers to the first 8-12 nucleotides upstream of PAM on the target DNA that are complementary to the corresponding sgRNA sequence. As used herein, the term “spacer” refers to nucleotide sequence in an sgRNA that is homologous to target nucleic acid sequence of interest in the targeted DNA molecule. As used here the term “Levenstein distance” refers to the minimum number of single base substitutions, insertions, or deletions required to change a sequence to another of the same length.

As used herein the term “multiplex” refers to multiple samples being processed at the same time. For example, “multiplex PCR” as used herein, refers to a technique whereby PCR is used to amplify several different DNA sequences simultaneously. As used herein the term “perfect” in relation to nucleic acid sequences refers to nucleic acid molecules comprising greater than 98%, preferably greater than 99%, preferably 100% homology to the designed nucleic acid sequence. In embodiments, the homology is determined by RNA sequencing as described herein.

### RNA Guided Sequence Enrichment

In one embodiment, the enrichment method comprises the use of RNA guided systems such as Cas9, Cas12, Cas13a, Cas13b, Cas12f, Cascade-Cas3, prokaryotic argonautes (*Marinitoga piezophila* (MpAgo), *Thermotoga profunda* (TpAgo), or *Rhodobacter sphaeroides* (RsAgo)) to selectively enrich a sequence population for sequences of interest.

In one aspect of the invention, the sequences of interest may be, for example, naturally occurring or synthetic genes, regulatory elements, pathways, entire genomes, oligonucleotides, gene circuits, cDNA libraries, viral vectors, exons, introns, origins of replication, retrotransposons, RNA-coding DNA, intergenic regions, plasmids, mitochondrial or chloroplast DNA, pseudogenes, DNA aptamers, ribozymes ss/dsDNA, ss/dsRNA, or ss/ds XNA (xeno nucleic acids).

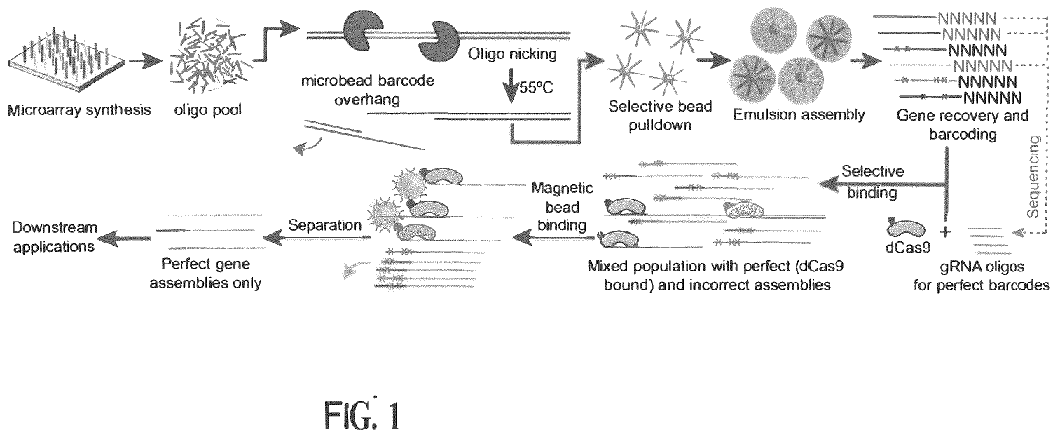

In one aspect, gene libraries comprising target sequences of interest may be assembled using emulsion PCR (see, e.g., U.S. Pat. No. 10,202,628 which is hereby incorporated by reference in its entirety) or via the DropSynth method (see, e.g., Plesa et al., Science 359, 343-347 (2018) which is hereby incorporated by reference in its entirety). The DropSynth method assembles genes through the isolation and assembly of microarray-derived oligonucleotides in droplets. As generally depicted in FIG. 1, sequences of interest, for example, genes, regulatory elements, pathways, or entire genomes, are bioinformatically split into multiple oligonucleotides and flanked with restriction sites, priming sequences and a 12-nt microbead barcode sequence that is common to all oligos needed to assemble a given sequence. Oligos are synthesized as a microarray-derived pool, amplified, and nicked using a nicking endonuclease, exposing each 12-nt microbead barcode as a single-stranded overhang. Nicked oligos are hybridized to a pool of barcoded microbeads that contain complementary 12-nt microbead barcode sequences, such that each bead pulls down all oligos for a particular assembly.

Bound beads are then encapsulated in droplets, where sequences are cleaved from the bead using a type IIS restriction enzyme and assembled into the sequences of interest by polymerase, preferably a high-fidelity polymerase such as KAPA HiFi, KAPA Robust, NEB Q5, Taq, Phusion, DeepVent, and others. Following assembly, the emulsions are broken, and sequence assemblies are recovered, purified and barcoded via ligation into a plasmid and directly PCR amplified. In some embodiments, the assemblies are next generation and the barcode corresponding to each assembly is mapped.

In some embodiments the sequence of interest may be a sub-pool or sub-population of a larger pool or sequence population. In one embodiment of this aspect of the invention, for example in some embodiments in which the DropSynth method is used, the sequences of interest to be enriched may be perfect assemblies. For example, 20 gene libraries of 1,536 genes were assembled using the DropSynth 2.0 method by Sidore et al. (see, Sidore et al., 2020, Nucleic Acids Research, Vol. 48, No. 16, which is hereby incorporated by reference in its entirety). Among genes with at least 100 barcodes Sidore et al. only achieved a median percent perfect assembly of 27.6% for one codon library and 22.6% for the second codon library.

In this aspect of the invention, barcodes corresponding to perfect sequences in the mixed population of perfect assemblies and those with errors and their relative distribution are mapped. Barcodes for perfect assemblies may be bioinformatically selected based on their relative representation, and to maximize the coverage of different genes while renormalizing their distribution to be more uniform.

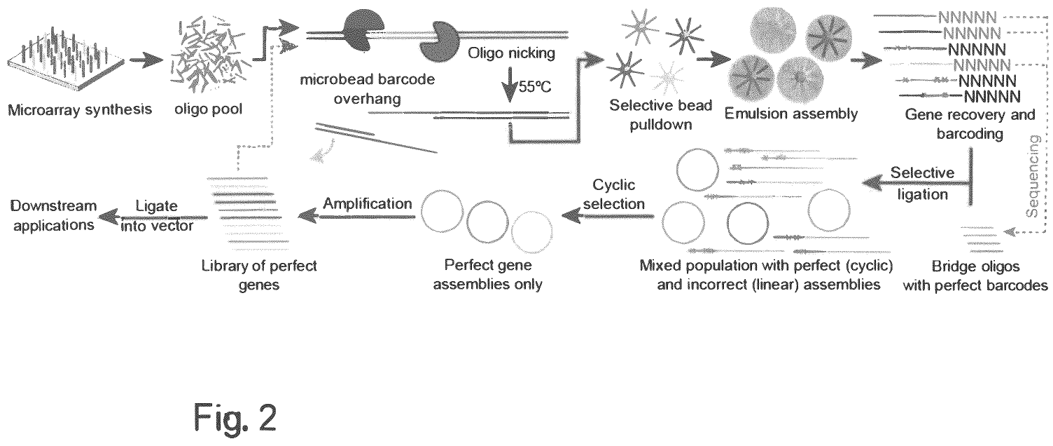

In one aspect of the invention, a population of nucleic acid molecules further enriched for perfect sequence assemblies is produced. In one embodiment of this aspect, a library of guide RNAs (“gRNAs”), particularly single guide RNAS (“sgRNAs”), targeted to DNA sequences of interest may be synthesized and complexed with an inactive Cas protein, such as dCas9. Streptococcus pyogenes CRISPR dCas9 RNA guided enrichment, as generally depicted in FIG. 2, may be used to enrich target sequences, such as perfect sequence assemblies, providing a scalable, multiplexed, sequence enrichment method.

In some embodiments, after assembly, sequences of interest to be enriched may be ligated into a vector comprising a protospacer adjacent motif (“PAM”) such as 5′-NGG-3′, and a bar code. Sequence. Barcoded amplicons may be sequenced using an Illumina MiSeq, PacBio Sequel II or Oxford Nanopore sequencer to map the corresponding barcode-linkages and determine library properties including percent perfects, required sequencing depth, coverage, and uniformity.

For such an enrichment method, a library of sgRNAs targeting multiple different sequences of inter may be provided. sgRNA library generation, however, is hindered by the cost, time, and protocol complexity required to produce sgRNA libraries specific for large numbers of unique target sequences. Thus, there remains a need for more simplified and less expensive methods for production of large-scale guide RNA libraries for guiding Cas9, such a deactivated Cas9, other CRISPR Cas systems or other RNA-guided enzymes to sequences of interest.

In one aspect of the invention described herein, improved methods for large-scale, multiplexed, guide RNA library production, particularly CRISPR guide RNA library production, such as CRISPR single guide RNA (sgRNA) library production is provided. Provided herein, in some embodiments, are improved methods for producing sgRNA oligonucleotides. In some embodiments, the improvement comprises reducing production costs and complexity, particularly for large-scale, multiplexed, sgRNA libraries.

Illustrative methods provided herein, reduce the cost of oligonucleotide synthesis by reducing the length of the oligonucleotides to be chemically synthesized in step 3 of FIG. 11 thus reducing the cost of an sgRNA library. Further, illustrative embodiments provided herein, reduce the sgRNA library cost by increasing the size of the oligonucleotide pool to be chemically synthesized by combining sub-pools for chemical synthesis into a larger pool. In some embodiments, the larger chemically synthesized pool may be separated into sub-pools by amplification of each sub-pool with sub-pool specific PCR primers.

Further, in some embodiments of this aspect of the invention, the methods described herein comprise producing a pooled sgRNA library, particularly libraries comprising sub-pools of sgRNAs.

Further, in some embodiments the methods described herein encompass the use of the described sgRNA pools in methods for targeted nucleic acid enrichment and/or cleavage of nucleic acid molecules, in multiplex, such as genome wide screening, gene synthesis, gene assembly and targeted sequencing. In some aspects of the invention, selected barcode sequences may be synthesized as a new oligo pool, sub-pool amplified, and used to synthesize single guide RNAs (“sgRNAs”), for each selected genetic assembly. In some embodiments of this aspect of the invention, sub libraries of original oligo pool having bar codes of interest may be sub pool amplified with sub pool specific primers without the need to synthesize a new oligo pool.

In some aspects of the invention, biotinylated dCas9 may be complexed with the target sequence population and specifically bind the barcodes of the target sequences of interest. In some embodiments, selectively bound target molecules of interest are isolated using Streptavidin (or other reactive linker) coated magnetic beads. These enriched libraries of target assemblies or subsequences may be next generation sequenced to determine enrichment factors and library metrics.

### Bridge Oligo Cyclization Mediated Enrichment

In some embodiments, a population of nucleic acid molecules enriched for perfect sequences, is produced as generally depicted in FIG. 1. In some embodiments, barcoded assemblies are sequenced after being assembled using the DropSynth method. Barcodes corresponding to perfect sequences in the mixed population and their relative distribution are mapped. Barcodes for perfect assemblies are bioinformatically selected based on their relative representation and maximize the coverage of different genes while renormalizing their distribution to be more uniform. DropSynth assembled molecules are processed to leave a blunt end immediately adjacent to the barcode region. In some embodiments, a new pool of short bridge 40-mer oligos are synthesized, where the new pool of bridge oligo nucleotides comprises the selected barcodes for perfect assemblies and a conserved region. Hybridization with these bridge oligos is used to selectively cyclize only the molecules containing a perfect sequence. These circular molecules are then selected (using enzymatic degradation of linear molecules or amplification of the circular molecules) and amplified to create a new library of perfect genes with a uniform distribution.

### EXAMPLES

### Example 1: In Vitro sgRNA Synthesis

### Double Stranded DNA Scaffold Oligonucleotide Preparation

Single stranded DNA oligonucleotides having the sequence GAGAACGGTCTCCTAGAAATAGCAAGTTAAAATAAGGCTAGTCCGTTATCAACTT GAAAAAGTGGCACC GAGTCGGTGCTTTT (SEQ ID No: 1) are annealed to their reverse complement (AAAAGCACCGACTCGGTGCCACTTTTTCAAGTTGA TAACGGACTAGCCTTATTTTAACTTGCTATTTCTAG GAGACCGTTCTC (SEQ ID No.: 2))

Briefly, 12.5 ul of a 100 uM solution of the scaffold oligonucleotide in IDTE Lab Ready Buffer (Integrated DNA Technologies, Coralville, IA), is mixed with 12.5 ul of a 100-uM solution of its reverse complement, also in IDTE LabReady buffer. 25 μl of Nuclease-Free IDT Duplex Buffer (Integrated DNA Technologies, Coralville, IA) is added and the 50 μl reaction is heated to 94° C. for 2 minutes in a thermocycler. The reaction is cooled to 25° C. for 45 minutes and then cooled to 4° C.

Prior to further use, the double stranded DNA is purified using a Monarch® PCR & DNA Cleanup Kit (New England Biolabs (Ipswich, MA) according to the manufacturer's instructions.

930 ng of dsDNA at a concentration of 37 ng/μl was produced.

### Spacer Oligonucleotide Design

Twenty base pair spacer sequences are computationally designed, as described in Example 5 below, for each sequence to be targeted by the sgRNA. The spacers are designed to omit the Bsa1 recognition site GAGACC (SEQ ID NO 3). Flanking sequences are appended to each spacer sequence to enable assembly and RNA transcription. For example, a T7 polymerase promoter sequence TTCTAATACGACTCACTATAG (SEQ ID NO 4), is appended to the 5′ end of each spacer sequence while a portion of the conserved sgRNA scaffold is appended to the 3′ end of each spacer and a Type II restriction site sequence TTTTAGAGCTAGAGGAGACCGTTCTC (SEQ ID NO 5) is appended to the 3′end of the conserved scaffold sgRNA sequence.

Orthogonal primer sets unique for each target sequence are selected and appended to the relevant spacer constructs to permit amplification of sub-pools of sequences. The forward sub pool orthogonal spacer is appended to the 5′ end of the T7 promoter and the reverse sub pool orthogonal to the 3′ end of the type II restriction site. As used herein, the term orthogonal primer set refers to primer pairs that do not interact with other primer pairs.

The designed spacer oligonucleotides are chemically synthesized as a single pool array of single stranded DNA by a commercial vendor such as Twist Bioscience, Agilent Technologies, CustomArray (GenScript Biotech), or LC Sciences. For example, in Table 1 below an orthogonal primer GGGTCACGCGTAGG (SEQ ID NO 6) is appended to the 5′ end of the T7 promoter of each spacer oligonucleotide in the pool, while the orthogonal primer GTGTGGCTGGGAAC (SEQ ID NO 7) is appended to the 3′ end of the sgRNA conserved scaffold sequence of each spacer oligonucleotide of the sub-pool to result in a 98 base pair oligonucleotide. The spacer oligonucleotides are sent to a commercial vendor for oligonucleotide library synthesis (“OLS”) as an array on a chip. Table 1 below shows the sequences of spacer oligonucleotides targeting 18 unique barcodes in the mCherry Red fluorescent protein (“RFP’) (750 bp) library, in which the wild type mCherry copies have at least 306,387 copies each of which is flanked by unique bar codes. Each bar code may have multiple physical copies.

TABLE 1

| | | SEQ |
|---|---|---|
| Sequence | | ID |
| Name | Sequence | No. |
| sgRNA 1 | GGGTCACGCGTAGGATTCTAATACGACTCACTATAGGCGTTCA | 8 |
| | ATTTAGATTAGGGTTTTAGAGCTAGAGGAGACCGTTCTCGTGT | |
| | GGCTGCGGAAC | |
| sgRNA 2 | GGGTCACGCGTAGGATTCTAATACGACTCACTATAGGCTATTG | 9 |
| | TTTCTAAAATGCGTTTTAGAGCTAGAGGAGACCGTTCTCGTGT | |
| | GGCTGCGGAAC | |
| sgRNA 3 | GGGTCACGCGTAGGATTCTAATACGACTCACTATAGGAGTGA | 10 |
| | GTTTAATCTGGGCGTTTTAGAGCTAGAGGAGACCGTTCTCGTG | |
| | TGGCTGCGGAAC | |
| sgRNA 4 | GGGTCACGCGTAGGATTCTAATACGACTCACTATAGGGATGAT | 11 |
| | AATGGCCCTGTTGTTTTAGAGCTAGAGGAGACCGTTCTCGTGT | |
| | GGCTGCGGAAC | |
| sgRNA 5 | GGGTCACGCGTAGGATTCTAATACGACTCACTATAGGAAACTT | 12 |
| | AGCGTAATATAAGTTTTAGAGCTAGAGGAGACCGTTCTCGTGT | |
| | GGCTGCGGAAC | |
| sgRNA 6 | GGGTCACGCGTAGGATTCTAATACGACTCACTATAGGTTCTGT | 13 |
| | GCTTAACAATGAGTTTTAGAGCTAGAGGAGACCGTTCTCGTGT | |
| | GGCTGCGGAAC | |
| sgRNA 7 | GGGTCACGCGTAGGATTCTAATACGACTCACTATAGGAGAGC | 14 |
| | GCCTCATTCATGTGTTTTAGAGCTAGAGGAGACCGTTCTCGTG | |
| | TGGCTGCGGAAC | |
| sgRNA 8 | GGGTCACGCGTAGGATTCTAATACGACTCACTATAGGTATTCG | 15 |
| | TATATGTGCGAAGTTTTAGAGCTAGAGGAGACCGTTCTCGTGT | |
| | GGCTGCGGAAC | |
| sgRNA 9 | GGGTCACGCGTAGGATTCTAATACGACTCACTATAGGACGGTG | 16 |
| | TCGCATTTGGAAGTTTTAGAGCTAGAGGAGACCGTTCTCGTGT | |
| | GGCTGCGGAAC | |
| sgRNA 10 | GGGTCACGCGTAGGATTCTAATACGACTCACTATAGGTTCGGA | 17 |
| | TCATGTCAACTTGTTTTAGAGCTAGAGGAGACCGTTCTCGTGT | |
| | GGCTGCGGAAC | |
| sgRNA 11 | GGGTCACGCGTAGGATTCTAATACGACTCACTATAGGATCTTA | 18 |
| | ATGAGTATTGATGTTTTAGAGCTAGAGGAGACCGTTCTCGTGT | |
| | GGCTGCGGAAC | |
| sgRNA 12 | GGGTCACGCGTAGGATTCTAATACGACTCACTATAGGTAGATA | 19 |
| | GACCTTTCACCGGTTTTAGAGCTAGAGGAGACCGTTCTCGTGT | |
| | GGCTGCGGAAC | |
| sgRNA 13 | GGGTCACGCGTAGGATTCTAATACGACTCACTATAGGAAGAG | 20 |
| | GTTTGCGATCTGCGTTTTAGAGCTAGAGGAGACCGTTCTCGTG | |
| | TGGCTGCGGAAC | |
| sgRNA 14 | GGGTCACGCGTAGGATTCTAATACGACTCACTATAGGTTATGA | 21 |
| | AAATTGTATGTCGTTTTAGAGCTAGAGGAGACCGTTCTCGTGTAr | |
| | GGCTGCGGAAC | |
| sgRNA 15 | GGGTCACGCGTAGGATTCTAATACGACTCACTATAGGATCCGA | 22 |
| | TTACGACAAATAGTTTTAGAGCTAGAGGAGACCGTTCTCGTGT | |
| | GGCTGCGGAAC | |
| sgRNA 16 | GGGTCACGCGTAGGATTCTAATACGACTCACTATAGGTCTAAT | 23 |
| | ACCGCACTCTCTGTTTTAGAGCTAGAGGAGACCGTTCTCGTGT | |
| | GGCTGCGGAAC | |
| sgRNA 17 | GGGTCACGCGTAGGATTCTAATACGACTCACTATAGGAACGCG | 24 |
| | TCCCGTTCATCGGTTTTAGAGCTAGAGGAGACCGTTCTCGTGT | |
| | GGCTGCGGAAC | |
| sgRNA 18 | GGGTCACGCGTAGGATTCTAATACGACTCACTATAGGTGACTC | 25 |
| | TGCTTAGCCTATGTTTTAGAGCTAGAGGAGACCGTTCTCGTGT | |
| | GGCTGCGGAAC | |

### OLS Oligonucleotide Sub-Pool Preparation

A 1/10 dilution of the commercial OLS chip pool is prepared according to the manufacturer's instructions. A mixtures of forward and reverse amplification primers for each sub-pool, is prepared, at a 10 μM final concentration of each primer and the spacer oligonucleotides are qPCR amplified as follows: A 50 uL reaction, for each single stranded spacer oligonucleotide is prepared, comprising: 1 uL template (1/10 OLS pool dilution), 25 uL forward amplification primer 10 uM, 1.25 uL reverse amplification primer, 10 uM, 21 uL UltraPure Distilled Water®, 25 uL NEB Q5 Master Mix (2×)®, 0.5 uL thiazole green 100×® is prepared, denatured for 45 sec at 98° C. in a thermocycler and cycled 35 times as follows:
15 sec 98° C. denaturation
5 sec 65° C. annealing and,
5 sec 72° C. extension.

Fluorescence is measured after each cycle and a 1-minute final extension is conducted at 72° C. After qPCR quantification, each sub-pool is amplified using Q5 DNA polymerase (New England Biolabs), 1 μL template (1/10 OLS pool dilution, 1.25 μL sub-pool specific forward primer 10 μM, 1.25 μL sub-pool specific reverse primer 10 μM, 21.5 μL UltraPure Distilled Water (Invitrogen) and 25 μL Q5 Hot Start PCR Master Mix (New England Biolabs)® for a total reaction volume of 50 μL.

The 50 μL reactions are initially denatured at 98° C. for 45 seconds and then cycled through the following incubation steps for the number of cycles determined by the above-qPCR protocol to arrive at the desired concentration:
5 sec 98° C. denaturation
5 sec 65° C. annealing.
15 sec 72° C. extension,
Followed by a 1-minute final extension at 72° C.

The amplified DNA oligonucleotides are then column purified using a Monarch DNA Clean-Up Kit (New England Biolabs) and run on an electrophoresis gel, size selected if necessary and quantified using QuBit Fluorometric Quantification (Thermo Fisher).

### Spacer Pool Oligonucleotide Template Preparation by Primer Extension

In one embodiment, double stranded spacer DNA oligonucleotides are prepared from single stranded DNA oligonucleotides by primer extension. Briefly, each of the sgRNA single stranded DNA oligonucleotides 1, 2, 3 and a single pool of sgRNA single-stranded DNA oligonucleotides 1 through 18 are extended with the GTTCCGCAGCCACC (SEQ ID NO 26) primer using Q5 2× Master Mix (New England Biolabs) in a thermocycler. Each reaction is denatured at 98° C. for 30 seconds, followed by annealing at 64° C. for 30 seconds and extension at 72° C. for 60 minutes.

The resulting double stranded DNA sgRNA oligonucleotides are purified, over 5 μg columns, using a Monarch DNA Clean Up Kit (New England Biolabs, Ispwich, MA) according to the manufacturer's instructions.

Assembly of sgRNA Spacer and Scaffold Oligonucleotides
Simultaneous Restriction Endonuclease Digestion and Ligation Assembly of sgRNA Spacer and Scaffold Oligonucleotides (Golden Gate Assembly)

A ratio of 2:1 of the 83 bp double-stranded DNA scaffold oligonucleotide to the 97 bp double-stranded spacer oligonucleotide is mixed with 10×T4 DNA Ligase Buffer (New England Biolabs) which has been supplemented 1:1 with 1 mM ATP, T4 DNA Ligase (New England Biolabs) and Bsal-HFv2 (New England Biolabs) and incubated in a thermocycler as follows: 5 minutes at 37° C.
20 minutes at 16° C.
20 minutes at 80° C.

The above incubation series is cycled for 100 cycles and then maintained at 12° C.

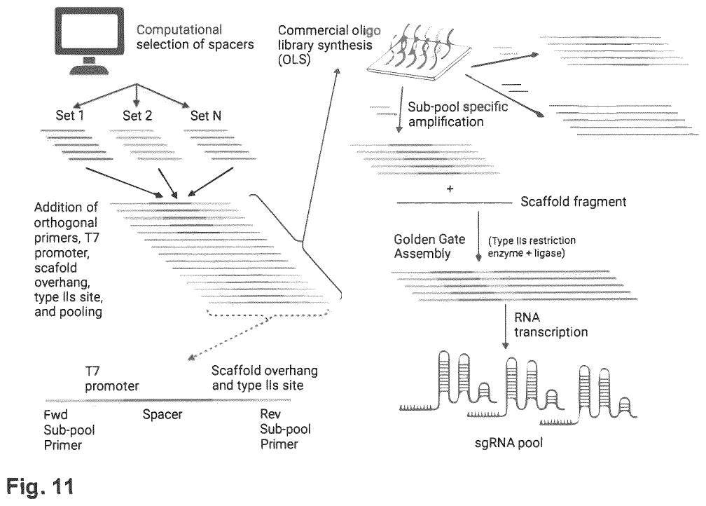

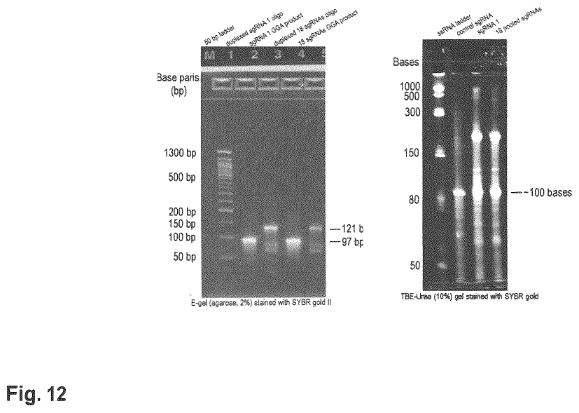

Digestion with Bsal-HFv2 results in a 4 bp 5′ overhang, TCTA (SEQ ID NO 27), on the spacer oligonucleotide that is complementary with the 5′ overhang, TAGA (SEQ ID NO 28), on the scaffold oligonucleotide which is also created by the Bsal-HFv2 digestion. The resulting assembled sgRNA double-stranded DNA molecules are run on an E-Gel (agarose, 2%) stained with SYBR gold II. FIG. 12 (left gel), shows that full length (˜120 bp) sgRNA oligonucleotides, lane 2 and lane 4, for the sgRNA 1 and the pooled sgRNA 1-18 oligonucleotide pool, respectively, were produced.

In Vitro Transcription of Assembled sgRNA Oligonucleotides

Three reactions, sgRNA 1, pooled 18 sgRNA, and the no template control (hereinafter “NTC”), are prepared as described in Table 2 below:

TABLE 2

| | | | 0 pmol | | 1 pmol DNA | | Final |
|---|---|---|---|---|---|---|---|
| Component | Volume | | Control | | input | | Concentration |
| NEB Murine Rnase | 0.5 | μl | 0.5 | μl | 0.5 | μl | 1 U/μl (20 |
| Inhibitor (NEB | | | | | | | units total) |
| M0134) | | | | | | | |
| DEPC-treated water | To fill | | 11.5 | μl | 5.5 | μl | |
| Golden Gate Products | Up to 8 μl | | N/A | | SgRNA 1 Golden | | |
| (full length sgRNA | | | | | Gate Product (6 μl, | | |
| oligos) abpt 121 bp | | | | | about 97 ng) | | |
| 1-5 pMol | | | | | SgRNA 18 pooled | | |
| sgRNA 1 GGA product | | | | | Golden Gate | | |
| (16.2 ng/ul) · sgRNA | | | | | Product (6 μl | | |
| 18 pooled GGA | | | | | (about 96 ng) | | |
| product (16 ng/ul) | | | | | | | |
| DTT (100 mM) | 2 | μl | 2 | μl | 2 | μl | 10 mM |
| NEB 10 X reaction mix | 2 | μl | 2 | μl | 2 | μl | 1X |
| (NEB B9012SVIAL) | | | | | | | |
| Ribonucleotide mix | 2 | μl | 2 | μl | 2 | μl | 8 mM each |
| (New England Biolabs, | | | | | | | rNTP |
| NEBN0466L) | | | | | | | |
| NEB T7 RNA | 2 | μl | 2 | μl | 2 | μl | |
| polymerase (NEB | | | | | | | |
| M0251L) | | | | | | | |
| Total Volume | 20 | μl | 20 | μl | 20 | μl | |

The samples are treated with DNAse 1 (NEB M0303) according to the manufacturer's protocol with the reaction components listed in Table 3 below:

TABLE 3

| Component | |
|---|---|
| Transcribed sgRNA | 20 μl reaction |
| DNAse 1 Reaction Buffer (10X) | 5 μl |
| DNAse 1 (RNAse-free) | 0.5 μl (1 unit) |
| Nuclease-Free Water | 24.5 μl |
| | Total Volume: 50 μl |

The reactions are mixed well, spun down, and incubated at 37° C. for 10 minutes. The reactions are column purified with Monarch RNA Kit (NEB T2040) and eluted with 25 μl of nuclease-free water in nuclease-free Eppendorf tubes.

The concentrations of the transcribed RNA are assessed by Qubit HS RNA (Thermo Fisher Q32852). The 18 pooled sgRNAs had a concentration of 68 ng/μl (1.7 μg total) the sgRNA 1 had a concentration of 13/ng μl (325 ng total) and the NTC was too low to assess.

The sgRNAs are run on a TBE-Urea (10%) denaturing gel (Bio-Rad 4566036) to confirm sgRNA size. A 1× running buffer is prepared by diluting 50 mL 10×TBE buffer (Bio-Rad1610733) with 450 mL nanopure water 1. The sample buffer (Boston Bioproducts, Cat BP-1400) comprises 89 mM Tris, 89 mM boric acid, 2 mM EDTA, pH 8.0, 12% Ficoll, 0.01% bromophenol blue, 0.02% xylene cyanole FF, and 7M urea. The samples are prepared as described below and run on a Mini-PROTEAN electrophoresis system (Bio-Rad 1658004) at 200 V constant for 60 minutes.

All samples have 10 ul final volume. The low range ssRNA ladder (NEB N0364S) comprises 1 ul of ladder (about 100 ng) 9.5 ul water 3. and 2 ul sample buffer. The S. pyogenes NEB control sgRNA (IDT) comprises 1 ul (˜100 ng), 2.7 ul water and 2 ul sample buffer. The. sgRNA 1 sample comprises 8 ul (˜100 ng) and 2 ul sample buffer. The 18 pooled sgRNA sample comprises 1.5 ul of the sample, 6.5 ul water and 2 ul sample buffer. 6.

The gel is post-stained with 1:10,000 SYBR gold nucleic acid stain (Thermo Fisher S11494) and diluted for 30 minutes prior to imaging. The SYBR gold stain is diluted by adding 5 ul SYBR gold to 50 mL 1×TBE buffer. The gel is imaged manually with 1-2 s of exposure. FIG. 12 (right gel) is an image of the TBE-Urea gel (10%) showing the individual transcribed sgRNA 1 and a pool containing 18 sgRNAs (100 bp).

### RNA Sequencing

The 18 sgRNAs are RNA sequenced using the sequences in Table 4 below.

TABLE 4

| | | | Seq |
|---|---|---|---|
| Sequence Name | Sequence | Description | ID |
| RT_primer_YG | AGCATATATCCCGGTCTGGA NNNNNNNNNNNNNNNNNN | DNA | 29 |
| | AAAAGCACCGA | primer | |
| TSO_YG | GCTAATCATTGCAAGCAGTGGTATCAACGCAGAGTACATrGrGrG | Template | 30 |
| | | Switching | |
| | | Oligo | |
| | | (TSO) | |
| TSO_primer_YG | CATTGCAAGCAGTGGTATCAAC | DNA | 31 |
| | | primer | |
| gene_specific_ | AGCATATATCCCGGTCTGGA | DNA | 32 |
| primer_YG | | primer | |
| sgRNA_cDNA_gibson_ | TCCAGACCGGGATATATGCTtatgcggtgtgaaataccgcac | DNA | 33 |
| FWD_YG | | primer | |
| sgRNA_cDNA_gibson_ | CACTGCTTGCAATGATTAGCcggtatcattgcagcactgg | DNA | 34 |
| REV_YG | | primer | |
| sgRNA_cDNA_1 kbp_ext_ | AGAGTTCTTGAAGTGGTGGC | DNA | 35 |
| FWD_YG | | primer | |
| sgRNA_cDNA_1 kbp_ext_ | GTGTGGAATTGTGAGCGGAT | DNA | 36 |
| REV_YG | | primer | |

### Reverse Primer Annealing

The Template Switching RT Enzyme Mix (NEB Product number: M0466S) is briefly centrifuged to collect the solution to the bottom of tube, then placed on ice. The Template Switching RT Buffer is thawed at room temperature, vortexed. centrifuged briefly to collect the solution to the bottom of tube, and then placed on ice.

The primer annealing reaction is prepared as indicated in Table 5 below (on ice) in a 0.2 ml nuclease free PCR tube for the 18 sgRNAs and NTC:

TABLE 5

| | | |
|---|---|---|
| Reagent | Volume | Final Concentration |

| | | | |
|---|---|---|---|
| sgRNA | _{—} | ul | 10 ng-1 ug |
| RT_primer_YG (10 μM) | 1 | μl | 1 μM |
| dNTP (10 mM) | 1 | μl | 1 mM |
| Murine RNase Inhibitor | 0.5 | μl | |

| | | |
|---|---|---|
| Nuclease-free Water | To fill | — |

| | | | |
|---|---|---|---|
| Total Volume | 6 | μl | — |

The reaction is mixed by pipetting up and down at least 10 times and centrifuged briefly to collect the solution to the bottom of the tube. The reaction is incubated for 5 minutes at 70° C. in a thermocycler with the lid temperature set at ≥85° C., then held at 4° C. until the next step.

### Reverse Transcription (RT) and Template Switching

The Template Switching RT Buffer vortexed and then briefly spun down. The RT reaction is prepared as indicated in Table 6 below.

TABLE 6

| | | |
|---|---|---|
| | | Final |
| Reagent | Volume | Concentration |

| | | | |
|---|---|---|---|
| Template Switching RT | 2.5 | μl | 1X |
| Buffer (4X) | | | |
| TSO_YG (75 μM) | 0.5 | μl | 3.75 μM |
| RT Enzyme mix (10X) | 1 | μl | 1X |
| Total Volume | 4 | μl | — |

The reaction is mixed by pipetting up and down at least 10 times and then centrifuged briefly to collect solution to the bottom of the tube. 4 μl of the RT reaction mix (above) is combined with 6 μl of the primer annealing reaction. mixed well by pipetting up and down at least 10 times, and then centrifuged briefly to collect the solution to the bottom of the tube. The 10 μl combined reaction is incubated in a thermocycler for 90 minutes at 42° C., then 5 minutes at 85° C. and held at 4° C.

qPCR and PCR

The RT reaction is diluted 2-fold with water and 1 μl of the diluted cDNA is used in a subsequent 25 μl PCR reaction. For low abundant RNA targets, up to 2.5 μl of undiluted cDNA may be used in a 25 μl PCR reaction.

The qPCR reaction is assembled on ice as described in Table 7 below:

TABLE 7

| | | | Final |
|---|---|---|---|
| Components | Volume | | Concentration |
| Diluted template switching cDNA product | 1 | μl | — |
| Q5 Hot Start High-Fidelity Master Mix (2X) | 12.5 | μl | 1X |
| (NEB #M0494) | | | |
| TSO_primer_YG (10 μM) | 1.25 | μl | 0.5 μM |
| Gene_specific_primer_YG (10 μM) | 1.25 | μl | 0.5 μM |
| H2O | 8.75 | μl | — |
| Thiazole green (100X) | 0.25 | | — |
| Total Volume | 25 | μl | — |

The qPCR reaction is mixed by pipetting up and down at least 10 times, and then centrifuge briefly to collect solution to the bottom of the tube. The reaction is then incubated in a thermocycler with the lid temperature set at ≥100° C., and qPCR is conducting as described in Table 8 below:

TABLE 8

| Step | Temperature | Time | | Cycles |
|---|---|---|---|---|
| Initial Denaturation | 98° C. | 30 | sec | 1 |
| Denaturation | 98° C. | 10 | sec | 5 |
| Annealing & | 72° C. | 5 sec (30 | | |
| Extension | | sec/kb) | | |
| Denaturation | 98° C. | 10 | sec | 5 |
| Annealing & | 70° C. | 5 sec (30 | | |
| Extension | | sec/kb) | | |
| Denature | 98° C. | 10 | sec | 40 (25-35 |
| | | | | recommended) |
| Annealing | 65° C. | 15 | sec | |
| Extension | 72° C. | 5 sec (30 | | |
| | | sec/kb) | | |
| Final Extension | 72° C. | 5 | min | 1 |

| | | |
|---|---|---|
| Hold | 4° C. | ∞ |

The PCR reaction is assembled on ice as described in Table 9 below.

TABLE 9

| | | | | | Final |
|---|---|---|---|---|---|
| Components | Volume | | 10x | | Concentration |
| Diluted template switching cDNA product | 1 | μl | 10 | μl | — |
| Q5 Hot Start High-Fidelity Master Mix (2X) | 12.5 | μl | 125 | μl | 1X |
| (NEB #M0494) | | | | | |
| TSO_primer_YG (10 μM) | 1.25 | μl | 12.5 | μl | 0.5 μM |
| Gene_specific_primer_YG (10 μM) | 1.25 | μl | 12.5 | μl | 0.5 μM |
| H2O | 8.75 | μl | 87.5 | μl | — |
| Total Volume | 25 | μl | 250 | μl | — |

The PCR reaction is mixed by pipetting up and down at least 10 times, and then centrifuged briefly to collect the solution to the bottom of the tube. The PCR reaction is incubated in a thermocycler with the lid temperature set at ≥100° C., and PCR is performed with the cycling conditions indicated in Table 10 below.

TABLE 10

| Step | Temperature | Time | | Cycles |
|---|---|---|---|---|
| Initial Denaturation | 98° C. | 30 | sec | 1 |
| Denaturation | 98° C. | 10 | sec | 5 |
| Annealing & | 72° C. | 5 sec (30 | | |
| Extension | | sec/kb) | | |
| Denaturation | 98° C. | 10 | sec | 5 |
| Annealing & | 70° C. | 5 sec (30 | | |
| Extension | | sec/kb) | | |
| Denature | 98° C. | 10 | sec | Cycle number |
| | | | | determined by qPCR |
| Annealing | 65° C. | 15 | sec | |
| Extension | 72° C. | 5 sec (30 | | |
| | | sec/kb) | | |
| Final Extension | 72° C. | 5 | min | 1 |

| | | |
|---|---|---|
| Hold | 4° C. | ∞ |

The PCR product is stored at −20° C. The PCR reaction yield is determined using HS DNA Qubit. The samples are run on a 4% E-gel to check the size of the products (about 186 bp products). 2 ul NEB 50 bp ladder (100 ng/ul) plus 18 ul water (total 200 ng) is loaded onto the E-gel along with 100 ng of the 18 sgRNA cDNAs.

### Vector Preparation for Gibson Assembly

A qPCR reaction is assembled as indicated in Table 11 below and as PCR is performed as described in Table 12 below.

TABLE 11

| | | | Final | |
|---|---|---|---|---|
| Component | 25 μl Reaction | | Concentration | |
| Q5 Hot Start High-Fidelity 2X Master | 12.5 | μl | 1X | |
| Mix | | | | |
| 10 μM | 1.25 | μl | 0.5 | μM |
| sgRNA_cDNA_gibson_FWD_YG | | | | |
| 10 μM | 1.25 | μl | 0.5 | μM |
| sgRNA_cDNA_gibson_REV_YG | | | | |
| Bayou Biolabs pUC19 (100 ng/ul) | 2 | ul | <5 | ng |
| Nuclease-Free Water | 8 | μl | | |

TABLE 12

| | | |
|---|---|---|
| STEP | TEMP | TIME |

| Initial Denaturation | 98° | C. | 30 | seconds |
|---|---|---|---|---|
| 25 Cycles | 98° | C. | 10 | seconds |
| | 67° | C. | 30 | seconds |
| | 72° | C. | 45 seconds | |
| | | | (20-30 sec/kb) | |
| Final Extension | 72° | C. | 2 | minutes |
| Hold | 4-10° | C. | | |

The DNA is column cleaned using Monarch DNA clean-up kit according to the manufacturer's instructions. The DNA is eluted in 26 ul nuclease-free water. DNA yield is measured using HS DNA Qubit according to manufacturer's instructions.

The DpnI digestion reaction for pUC19 template is assembled as described in Table 13 below.

TABLE 13

| Component | 50 μl Reaction | |
|---|---|---|
| PCR product (1 μg) | 25 | ul |
| 10X cutmark Buffer | 5 μl | (1X) |
| DpnI | 1.0 μl | (20 units) |
| Nuclease-free Water | 19 | μl |

The digestion reaction is incubated for 15 minutes. The DNA is cleaned using a Monarch DNA clean-up kit. The DNA is eluted in 26 ul nuclease-free water. The yield is measured using HS DNA Qubit. 2 ul NEB 50 bp ladder (100 ng/ul) plus 18 ul water (200 ng total) is loaded and run on a 1% E-gel along with 100 ng of the 18 sgRNA cDNAs and sgRNA size is determined (about 1.5 kb fragment).

Gibson Assembly of Digested pUC19 and sgRNA cDNA

Assemble the Gibson assembly reaction as indicated in Table 14 below.

TABLE 14

| | | | Positive | | Negative | |
|---|---|---|---|---|---|---|
| | Assembly | | Control | | Control | |
| pUC19 fragment (100 ng) | _{—} | μl | — | | _{—} | μl |
| sgRNA cDNA (42.07 ng) | _{—} | μl | — | | — | |
| NEBuilder ® Positive Control | — | | 10 | μl | — | |
| Gibson Assembly Master Mix | 10 | μl | 10 | μl | 10 | μl |
| (2X) | | | | | | |
| Deionized H2O | To fill | | — | | To fill | |
| Total Volume | 20 | μl | 20 | μl | 20 | μl |

Incubate samples in a thermocycler at 50° C. for 15 minutes. The DNA is cleaned using Monarch DNA clean-up kit. The DNA is eluted in 12 ul elution buffer and DNA yield is measured using HS DNA Qubit.

### PCR Fragment for Sequencing

A qPCR reaction is assembled as indicated in Table 15 below and as PCR is performed as described in Table 16 below.

TABLE 15

| | | | Final |
|---|---|---|---|
| Component | 25 μl Reaction | | Concentration |
| Q5 Hot Start High-Fidelity 2X Master | 12.5 | μl | 1X |
| Mix | | | |
| 10 μM | 1.25 | μl | 0.5 μM |
| sgRNA_cDNA_1kbp_ext_FWD_YG | | | |
| 10 μM | 1.25 | μl | 0.5 μM |
| sgRNA_cDNA_1kbp_ext_REV_YG | | | |
| Gibson Assembly Product | _{—} | ul | 1 ng-1 ug |

| | |
|---|---|
| Nuclease-Free Water | To fill |

TABLE 16

| | | |
|---|---|---|
| STEP | TEMP | TIME |

| Initial Denaturation | 98° | C. | 30 | seconds |
|---|---|---|---|---|
| 25 Cycles | 98° | C. | 10 | seconds |
| | 65° | C. | 30 | seconds |
| | 72° | C. | 25 seconds | |
| | | | (20-30 sec/kb) | |
| Final Extension | 72° | C. | 2 | minutes |
| Hold | 4-10° | C. | | |

The DNA is cleaned using a Monarch DNA clean-up kit and eluted in 21 ul. The yield is measured using HS DNA Qubit and 2 ul of the NEB 1 kb plus ladder (100 ng/ul), 200 ng total, 50 ng of, the pUC19 fragment (about 1576 bp), 50 ng of the cDNA (about 186 bp), 20 ng of the Gibson assembly product (about 1766 bp) and 50 ng of PCR product About 1014 bp) are run on a 2% E-gel.

10 ul or more of a 30 ng/ul PCR product (1014 bp) sample is provided to a commercial vendor, Plasmidsaurus (Oxford Nanopore Sequencing) for sequencing and 2 GB data is requested.

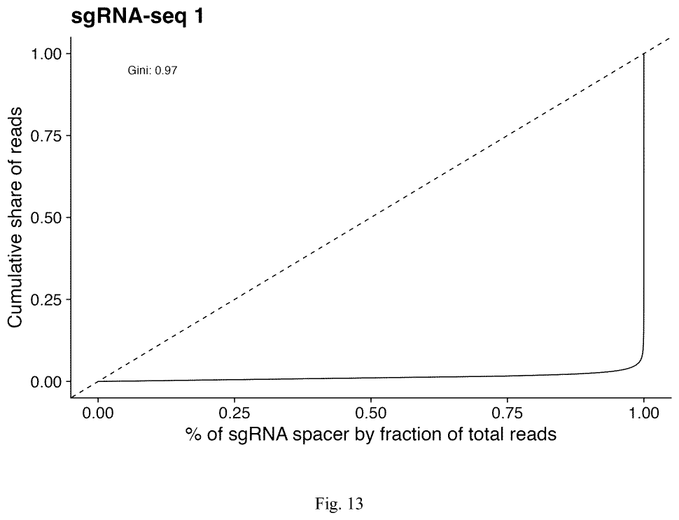

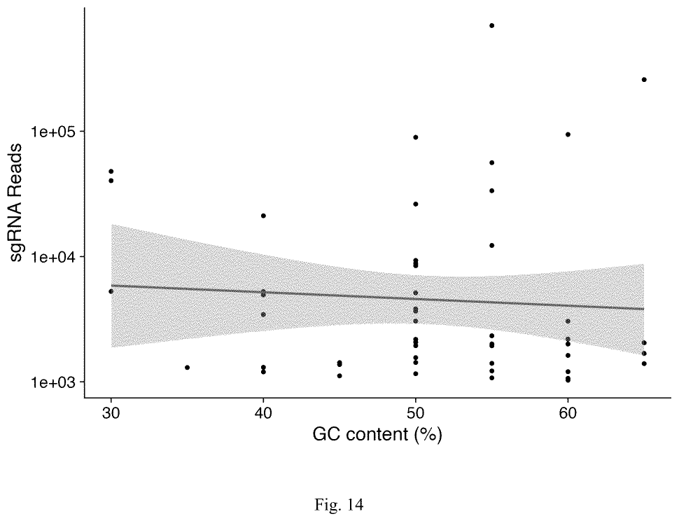

RNA sequencing validated that all 18 sgRNAs were made and was also used to analyze the sgRNA distribution. FIG. 13 is a Lorenz curve of the sgRNA distribution (Gini coefficient 0.97) indicating that while all 18 sgRNA were produced there was a non-uniform distribution. The plot of FIG. 14 indicates that for all spacer oligonucleotides (perfect and those with errors) with at least 1000 reads, there is little correlation between the spacer oligonucleotide GC content and oligonucleotide distribution.

### Example 2: dCas9 Target Sequence Enrichment

Ribonucleoprotein complexes (RNPs) consisting of the dCas9 protein complexed to single guide RNAs were prepared. sgRNAs provide targeted specificity upon Cas9 recognition of the three-nucleotide proximal PAM on the complementary DNA non-target strand.

18 sgRNAs are synthesized with Golden Gate Assembly as described above, diluted to 300 nM. in Nuclease free water and stored on ice. dCas9-3×FLAG-Biotin Protein (Sigma-Aldrich cat: DCAS9PROT-50UG) was diluted to 1 uM according to the manufacturer's instructions and stored on ice.

The RNP pools were diluted in PCR tubes by adding reagents in the order listed in Table 17 below and stored on ice.

TABLE 17

| | Enrichment | | Enrichment RNPs (for | |
|---|---|---|---|---|
| Condition | RNPs | | no template control) | |
| RNP formation ratio (dCas9 | 3:1 | | 3:1 | |
| to sgRNAs) | | | | |
| Component | | | | |
| Murine RNase inhibitor | 0.75 | ul | 0.75 | ul |
| (NEB cat: M0314S) | | | | |
| Nuclease-free water to fill | 19.25 | ul | 19.25 | ul |
| 10XNEBuffer 3.1 (NEB cat: | 3 | ul | 3 | ul |
| B6003S) | | | | |
| 18 pooled sgRNAs (300 nM) | 3 | ul | 3 | ul |
| dCas9-Biotin 1 uM | 1 | ul | 1 | ul |
| Total vol: | 27 | ul | 27 | ul |

Samples were mixed, pulse-spun in a microfuge, pre-incubated at 25° C. in a thermocycler for 10 minutes and then incubated at 37° C. for 10 minutes. The target sequence library of mCherry Red Fluorescent Protein (“RFP”) having the DNA sequence: atgtttccaagggcgaggaggataacatggctatcattaaagagttcatgcgcttcaaagttcacatggagggttctgttaacggtcacgagttcgagatcgaag gcgaaggcgagggccgtccgtatgaaggcacccagaccgccaaactgaaagtgactaaaggcggcccgctgccttttgcgtgggacatcctgagcccgcaatt tatgtacggttctaaagcgtatgttaaacacccagcggatatcccggactatctgaagctgtcttttccggaaggtttcaagtgggaacgcgtaatgaattttgaaga tggtggtgtcgtgaccgtcactcaggactcctccctgcaggatggcgagttcatctataaagttaaactgcgtggtactaattttccatctgatggcccggtgatgca gaaaaagacgatgggttgggaggcgtctagcgaacgcatgtatccggaagatggtgcgctgaaaggcgaaattaaacagcgcctgaaactgaaagatggcg gccattatgacgctgaagtgaaaaccacgtacaaagccaagaaacctgtgcagctgcctggcgcgtacaatgtgaatattaaactggacatcacctctcataatga agattatacgatcgtagagcaatatgagcgcgcggagggtcgtcattctaccggtggcatggatgagctgtacaaataa (SEQ ID NO 37) was prepared by PCR-amplifying the wild type mCherry 750 bp RFP gene and ligating it into the pEVBC3 vector which is a pUC19 derived vector. The pEVBC3 vector added PAM and a unique 20-mer barcodes to each copy of the wild type gene. The ligated vector was transformed into competent E. coli and the resulting colonies harvested, mini-prepped, and the MiSeq system was used to obtain barcode reads.

The mCherry RFP library was diluted 2:1 with water and the enrichment reaction was prepared as indicated in Table 18 below.

TABLE 18

| | | No template control |
|---|---|---|
| Condition | Enrichment | (NTC) |
| RNPs | 27 ul | 27 ul |
| EVBC3-RFP scrape DNA (50 | 1 ul | N/A |
| ng/ul) | | |
| Nuclease-free water to fill | 2 ul | 3 ul |

Samples were mixed thoroughly, pulse-spun in a microfuge and incubated for 15 minutes at 37° C.

Dynabeads M-270 Streptavidin (Thermo Scientific cat: 65306) were prepared and added to enrichment reactions as described below, to bind biotin on dCas9 to pull-down the dCas9 bound to DNA sequences having the targeted barcodes.

Dynabeads were washed 1× in binding and wash buffer (B&W) by diluting 2×B&W buffer (10 mM Tris-HCl, 1 mM EDTA·Na₂, pH 7.5, 2M NaCl) 2-fold with nanopure water. 5 ul of beads were washed per enrichment reaction (including NTC) according to the manufacturer's instructions as follows:
stock bottle of beads was vortexed for 30 seconds and then the beads were transferred to a 1.5 mL tube.
1 ml of 1×B&W buffer was added to the beads.
The tube was placed on a magnetic rack for 1 minute and the supernatant was discarded.
The beads were washed with a 1×B&W buffer.
Steps c. and d. were repeated three times.
After the final wash, the beads were resuspended in 2×B&W buffer.

10 ul of the washed beads were added to each enrichment reaction and the resulting reaction mixture was transferred to a 1.5 mL tube and incubated in a thermomixer at 1700 rpm and 37° C. for 30 min. The enrichment reaction and beads (including NTC) were washed to remove non-target DNA prior to PCR amplification and sequencing.

Briefly, the enrichment reaction beads were spun down and transferred to fresh 5 mL tubes. The enrichment reactions and beads were washed according to Table 19 below.

TABLE 19

| | Number of | 2X B&W wash | |
|---|---|---|---|
| Reaction | washes | volume | |
| Enrichment | 9 | 2 | mL |
| NTC | 6 | 50 | l |

Washes were conducted as follows: The 5 mL tubes were placed on magnetic rack for 1 minute. The supernatant was discarded. The beads were resuspended in the appropriate 2×B&W wash volume and before the sixth wash, the beads and buffer were transferred into fresh 5 ml tubes. After washing, each sample was resuspended in 60 us nuclease-free water.

Quantitative polymerase chain reactions (qPCR) of enrichment samples were conducted to determine the number of cycles needed to amplify enrichment DNA products and avoid overamplification. qPCR reactions for each template were prepared in duplicate. For enrichment and NTC templates, the samples were pipetted up and down to resuspend magnetic beads. The qPCR of the samples was prepared as indicated in Table 20 below using Q5 2× Master mix (NEB cat: M0492) and thiazole green dye diluted to 100× (Biotium cat: 40086).

TABLE 20

| Component | 1x reaction (50 ul reactions) | |
|---|---|---|
| Water to fill | 9.5 | ul |
| Q5 2X Master mix | 25 | ul |
| Forward primer: | 2.5 | ul |
| Mi7_FWD_Amp_NV (10 uM) | | |
| Reverse primer: | 2.5 | |
| Mi8_REV_Amp4_Index2_NV | | |
| (10 uM) | | |
| Enrichment/beads | 10 ul (add after aliquoting if you | |
| | are making a master mix) | |
| Thiazole green (100X) | 0.5 | ul |

qPCR was performed as described in Table 21 below. The resulting qPCR product was ˜600 bp that spans the barcode region.

TABLE 21

| Cycles | Step | Temp | Time | |
|---|---|---|---|---|
| 1 | Denaturation | 98° C. | 30 | seconds |
| 40 | Denaturation | 98° C. | 10 | seconds |
| | Annealing | 72° C. | 30 | seconds |
| | Extension | 72° C. | 2 | minutes |

The enrichment products were bulk amplified by performing PCR as described in Table 22. 4×PCR reactions per enrichment sample and one PCR for the NTC were prepared.

TABLE 22

| | 1x reaction (50 | |
|---|---|---|
| Component | ul reactions) | |
| Water to fill | 10 | ul |
| Q5 2X master mix | 25 | ul |
| Forward primer: Mi7_FWD_Amp_NV (10 | 2.5 | ul |
| uM) | | |
| Reverse primer: | 2.5 | ul |
| Mi8_REV_Amp4_Index2_NV (10 uM) | | |
| Enrichment/bead washes | 10 | ul |

PCR was performed for 24 cycles as described in Table 23 below.

TABLE 23

| Cycles | Step | Temp | Time | |
|---|---|---|---|---|
| 1 | Denaturation | 98° C. | 30 | seconds |
| 24 | Denaturation | 98° C. | 10 | seconds |
| | Annealing | 72° C. | 30 | seconds |
| | Extension | 72° C. | 2 | minutes |
| 1 | Final Extension | 72° C. | 2 | minutes |
| 1 | Hold | 10° C. | | |

Monarch DNA Clean-U Kit (NEB cat: T1030L) was used to purify PCR products. The clean-up was started by adding the appropriate amount of binding buffer and pooled PCR products into a 1.5 mL tube. The tube was then placed in magnetic binding rack for one minute. After which the supernatant was transferred to the 5 ug clean-up column and clean up proceeded according to the manufacturer's instructions.

A Qubit™ 1×dsDNA High Sensitivity (HS) assay kit (Thermo Scientific cat: Q33231) was used to quantify the enrichment products. 81.0 ng/ul (1.215 ug total) of the enrichment products and 43.2 ng/ul (648 ng total of the NTC) was obtained.

The ˜600 bp PCR products were gel extracted using Invitrogen™ E-Gel™ CloneWell™ II Agarose Gel, 0.8% (Thermo Scientific cat: G661818). About 500 ng of PCR DNA product was loaded onto the gel along with the NTC as a negative control.

The following samples were loaded onto rows 1, 5-6 and the gel was run according to the manufacturer's instructions:
Lane 1:1 kb plus ladder (NEB), 0.5 ul (˜500 ng) ladder, ul nuclease-free water and 2.5 ul 2× sample loading buffer (Lanes 2-4 are unrelated)
Lane 5: Enrichment product, 6.2 ul (˜500 ng) product, 16.3 nuclease free μl water, and 2.5× loading buffer
Lane 6: 11.6 ul (˜500 ng) NTC, 10.9 μl water and 2.5 2.x loading buffer

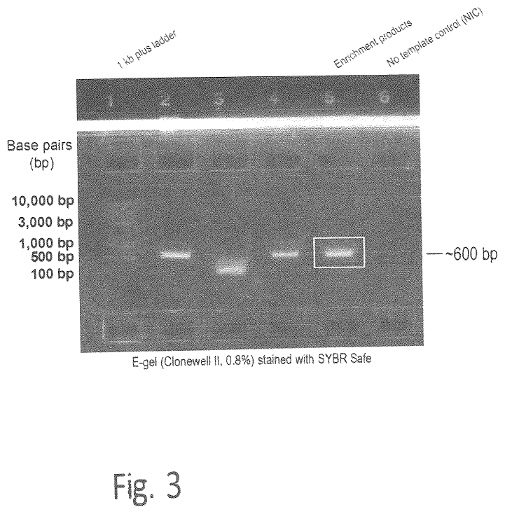

FIG. 3 shows the E-gel (Clonewell 11, 0.8%) stained with SYBR Safe. The enrichment products in lane 5 are about 600 base pairs long.

After gel extraction, the gel extracted products were pooled and concentrated using a Monarch DNA Clean-Up Kit (New England Biolabs, Ipswich, MA) The purified products were eluted and quantified using the Qubit DNA Quantification 1×HS DNA Kit and the enriched products were sequenced by next generation sequencing (“NGS”) and analyzed.

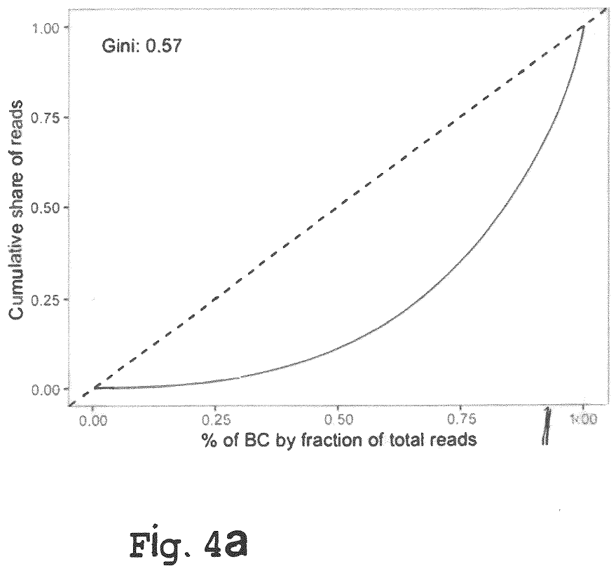

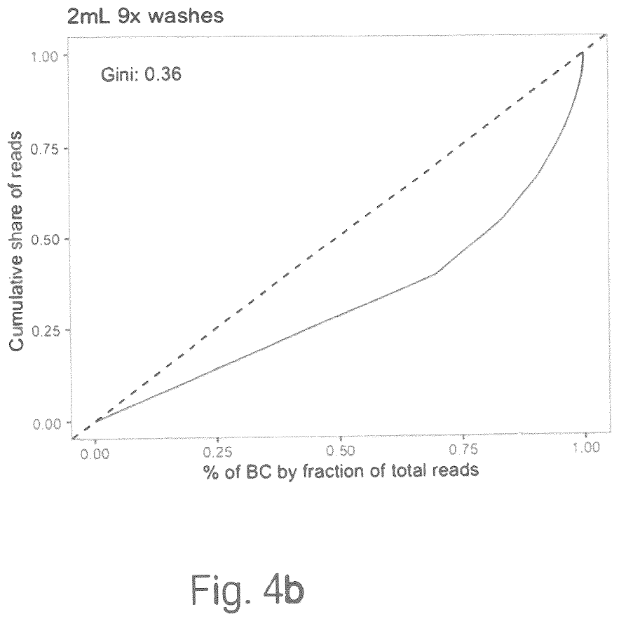

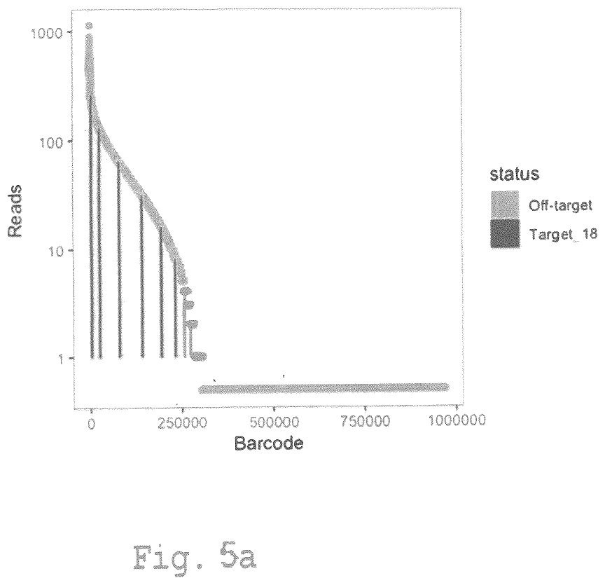

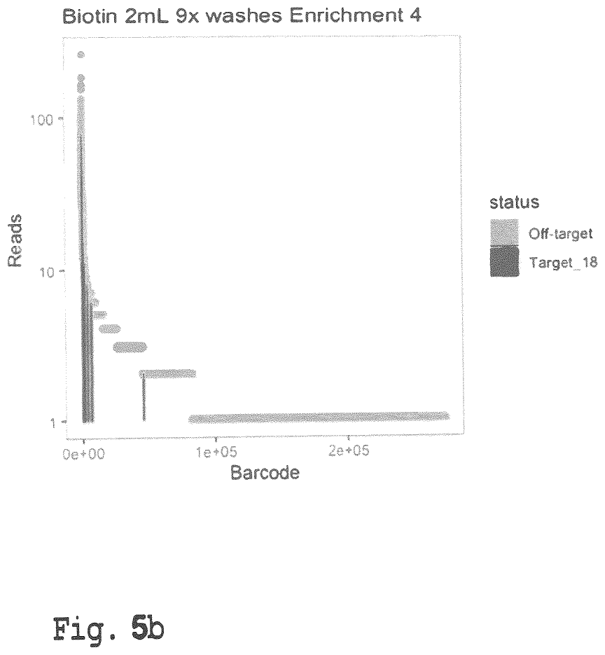

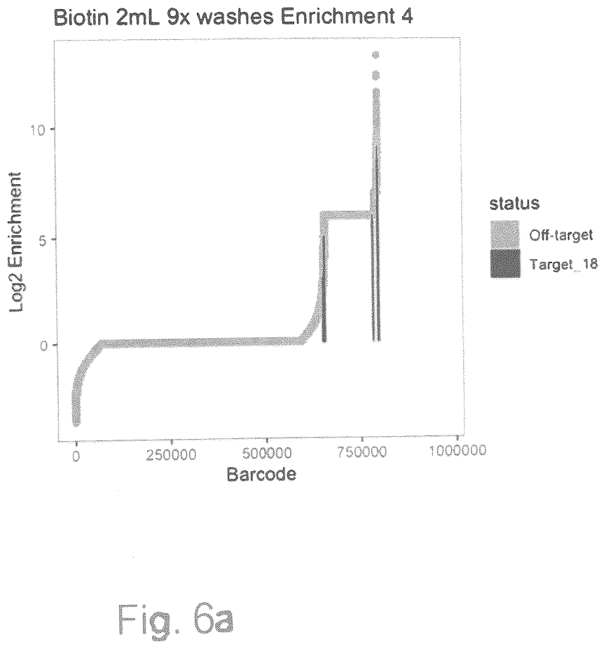

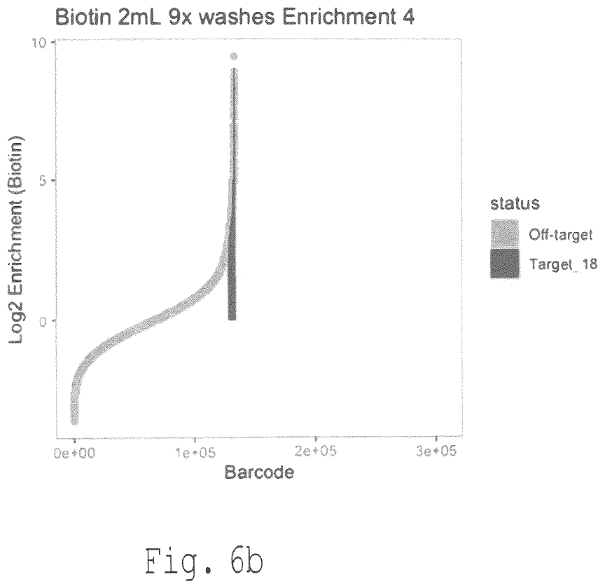

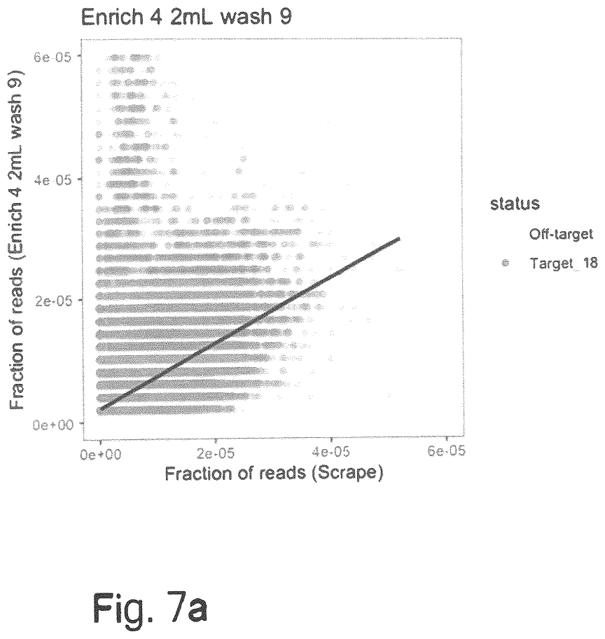

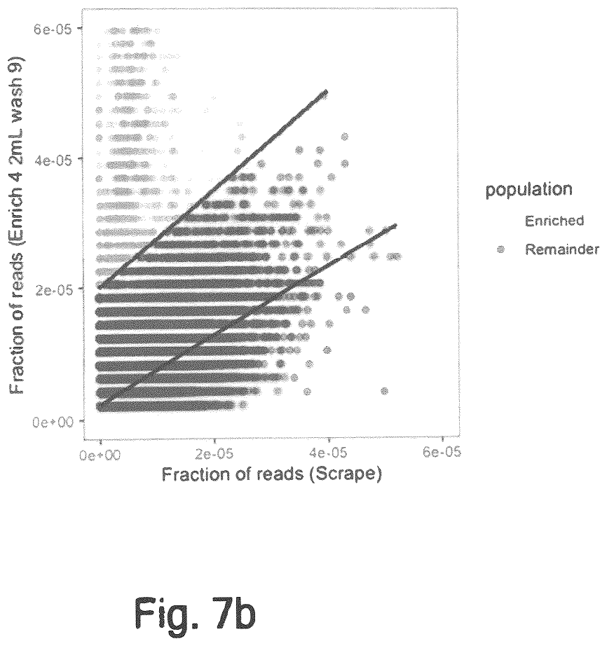

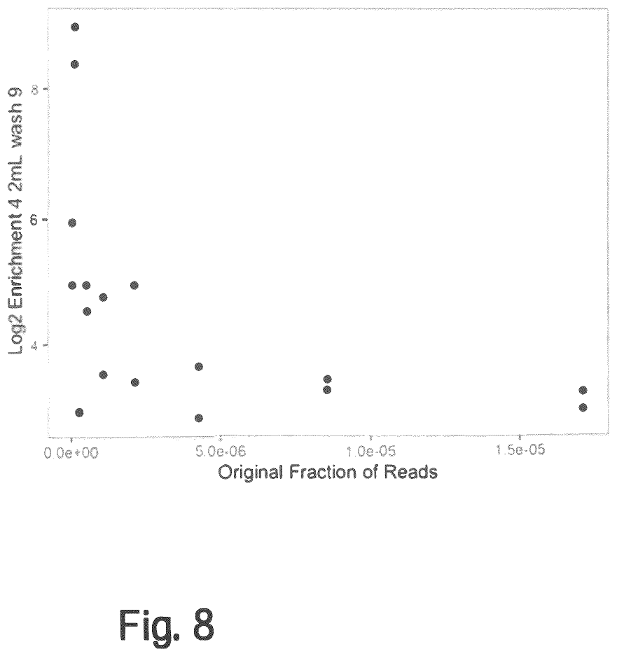

FIG. 4a shows the Lorenz curve and Gini coefficient for the unenriched mCherry RFP library while FIG. 4b shows the same for enriched library after nine 2 mL washes. FIG. 5a shows the number of reads for each selected barcode observed, (rank ordered in blue) for the unenriched mCherry RFP library while FIG. 5b shows the same for enriched library after nine 2 mL washes. FIG. 6a shows the Log 2 fold enrichment of the mCherry RFP library (on target in blue and off target in red) The horizontal line is a result of barcodes that were not observed at all in the original library sequencing (assigned 0.5 reads) but that were observed in the enriched library with one read. FIG. 6b shows the same data as FIG. 6a except that the low read barcodes have been removed for clarity. The vertical blue lines show the Log 2 enrichment of on target sequencing reads. FIG. 7a shows the correlation between the fraction of barcode sequencing reads for the unenriched library (x-axis) vs the fraction of reads for the enriched library (y-axis). FIG. 7b is the same plot as FIG. 7a except with the fraction of reads for the enriched reads in red (y axis) separated from the fraction of barcode reads in blue (x-axis). FIG. 8 shows the Log 2 barcode enrichment observed for the enriched mCherry RFP library (y-axis) as it corresponds to the original barcode population fraction (x-axis).

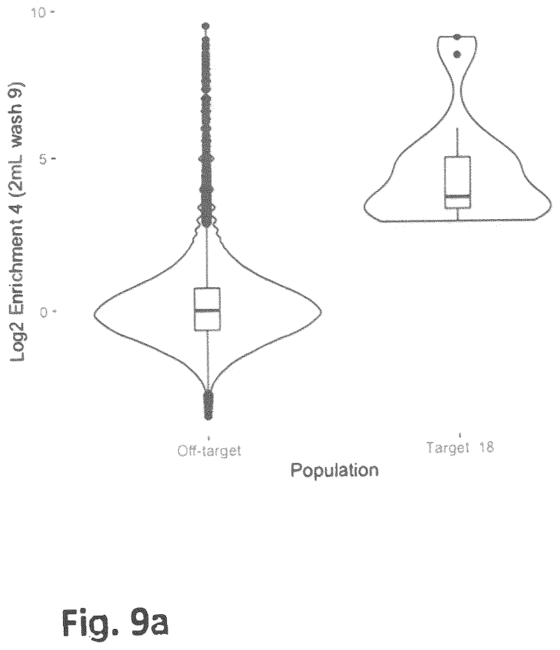

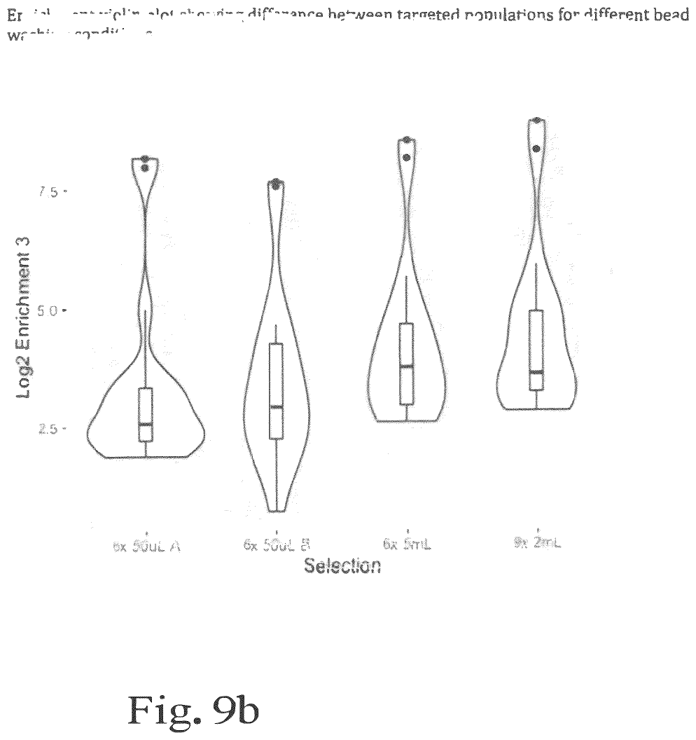

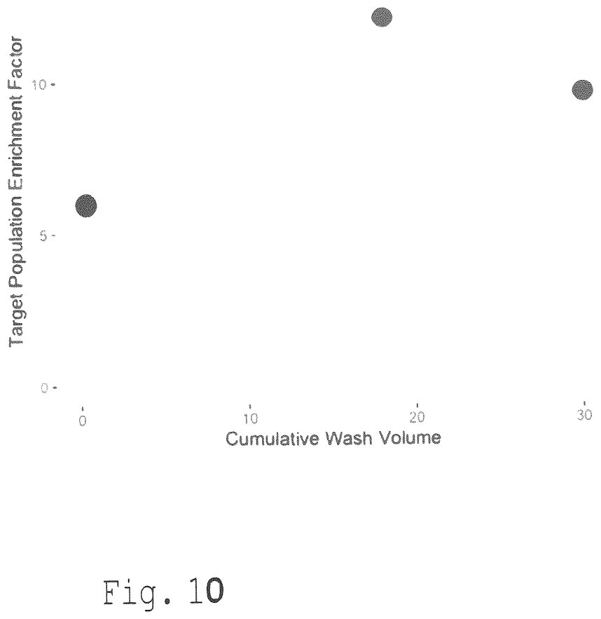

FIG. 9a is a violin plot showing the difference between targeted and untargeted barcode populations in the mCherry RFP library. FIG. 9b is a violin plot showing the difference in Log 2 enrichment of the RFP library under different bead washing conditions (x-axis). FIG. 10 shows the total population barcode fraction enrichment (y-axis) as a function of the cumulative wash volume used to wash the bound beads (x-axis).

### Example 3: Comparison of Methods of Removal of DNA Removal from dCas9

The 18 sgRNAs synthesized in Example 1 above were again used to enrich the RFP library as described in Example 2 above along with a linear and supercoiled controls except that immediately following enrichment but prior to the wash protocol, all the pooled 18 enriched sub-pools and controls were tested under the following 3 DNA removal methods.
1. Boiling Treatment: Cas9 is heat inactivated by incubating at 65 C for 5 min. After enrichment treatment (sgRNA-dCas9 DNA binding incubation) the protocol was modified as follows:
   a. The enrichment treatment sample was transferred to a 1.5 ml microcentrifuge tube.
   b. The sample was incubated at 65° C. for 5 min in thermomixer (mixing at 1700 rpm).
   c. The magnetic rack was used to remove supernatant to be used as PCR template.
   d. All samples were stored at 4° prior to qPCR reaction.
2. Proteinase K Treatment:
   a. The enrichment treatment sample was transferred to 1.5 ml microcentrifuge tube,
   b. 1 ul proteinase K was added to sample, mixed, pulse-spun in a microfuge, and incubated for 10 min at RT while shaking in thermomixer at 1700 rpm,
   c. A magnetic rack was used to remove supernatant,
   d. The supernatant was cleaned using Monarch DNA clean-up kit and eluted in 50 ul hot elution buffer.
   e. The clean DNA was used as PCR template.
3. Urea Treatment:
   a. The enrichment treatment sample was transferred to 1.5 ml microcentrifuge tube,
   b. 200 ul of 8 M urea was added to the sample, mixed and pulse-spun in a microfuge. The samples were incubated for 30 min at RT (Aalipour et al) while shaking in thermomixer at 1700 rpm.
   c. A magnetic rack was used to remove supernatant.
   d. The supernatant was cleaned using Monarch DNA clean-up kit and eluted in 50 ul hot elution buffer.
   e. The clean DNA was used as PCR template.

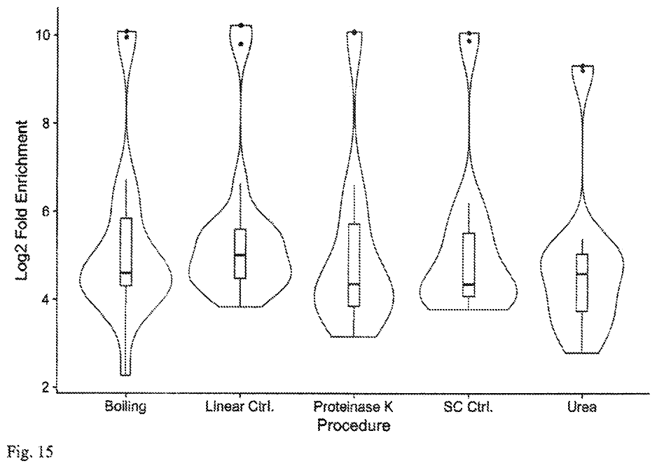

The results are given in the violin plot of FIG. 15 and Table 24 below where: the total population enrichment=(sum of all target fractions of reads after enrichment)/(sum of all target fractions of reads before enrichment), The median log 2 fold enrichment=median value of all the targets log 2 (fractions of reads after enrichment/fractions of reads before enrichment) and the off-target dropouts are how many barcodes have >1 reads before enrichment and 0 reads after enrichment.

TABLE 24

| | | | Total Number |
|---|---|---|---|
| | Total | Median | of Off-Target |
| | Population | log2 fold | Barcode |
| Sample | Enrichment | enrichment | Dropouts |
| Proteinase K | 23.25x | 4.36 | 131,254 |
| Boiling | 23.12x | 4.60 | 95,186 |
| Urea | 15.14x | 4.59 | 74,542 |
| Supercoiled control | 23.77x | 4.35 | 83,414 |
| Linear control | 27.65x | 5.00 | 106,119 |

Treatment with proteinase K maximized the total population enrichment and total number of off-target barcode dropouts.

### Example 4: Effect of Enrichment Incubation Time and Amount of Input Target Library DNA

Three of the 18 sgRNAs synthesized in Example 1 (sgRNA 7, sgRNA 8, and sgRNA 15 as in Table 1) above were ordered as synthetic RNA from a commercial vendor, IDT DNA, to remove confounding effects from sgRNA synthesis. These were used to enrich the RFP library as described in Example 2 above except that Proteinase K treatment as described in Example 3 was used and the enrichment reaction was run for the amount and time and with the amount of input target library DNA indicated in Table 25 below:

TABLE 25

| Sample Number | DNA input (ng) | Incubation Time (min) |
|---|---|---|
| 1 | 50 | 15 |
| 2 | 50 | 60 |
| 3 | 50 | 480 |
| 4 | 500 | 15 |
| 5 | 500 | 60 |
| 6 | 500 | 480 |

Table 26 below shows the effect of these conditions on Total Population Enrichment, Median log 2 fold enrichment and total number of off-target barcode dropouts.

TABLE 26

| | | | Total Number |
|---|---|---|---|
| | Total | Median | of Off-Target |
| | Population | log2 fold | Barcode |
| Sample | Enrichment | enrichment | Dropouts |
| 1-50 ng-15 min | 152x | 6.30 | 152,457 |
| 2-50 ng-60 min | 61x | 5.17 | 228,633 |
| 3-50 ng-480 min | 84x | 5.86 | 271,126 |
| 4-500 ng-15 min | 1228x | 9.39 | 133,410 |
| 5-500 ng-60 min | 397x | 7.85 | 141,463 |
| 6-500 ng-480 min | 341x | 7.52 | 57,661 |

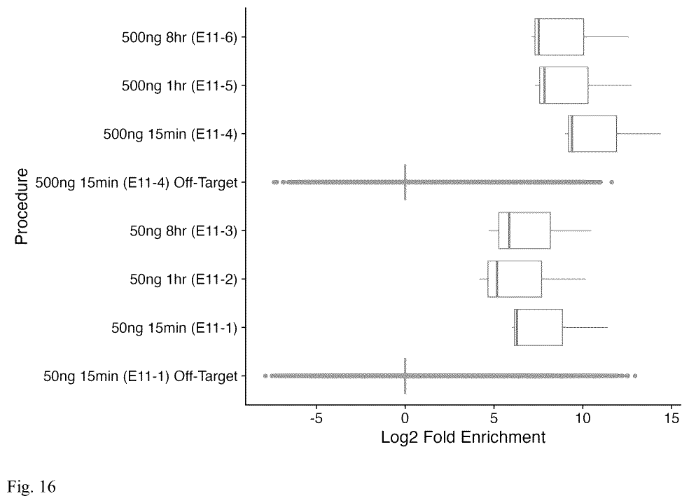

As Table 26 illustrates, increasing the amount of input DNA resulted in a significant increase in the observed enrichment metrics, while shorter (15 minutes) rather than longer incubation times (>=60 min) had a more beneficial effect on enrichment. This data is also graphically illustrated in the boxplot of FIG. 16.

### Example 5: Large Scale Pooled In Vitro RNA Synthesis

Barcode protospacers from within DropSynth synthesized gene libraries containing 384 to 1536 homologs of Dihydrofolate reductase (DHFR) enzymes are computationally selected.

### Computational Selection

Briefly, the computational algorithm is populated with 3 sets of data 1) the raw sequencing reads (linking genes and barcodes), 2) the sequence of the plasmid vector containing the sequence of interest. (Targeting plasmid vector sequences is to be avoided, and 3) either the DNA or amino acid sequences of the sequences of interest.

The algorithm extracts the barcode sequence and the gene sequence based on the conserved sequences flanking each. This produces a set of gene-barcode pairs which were successfully mapped and a set which failed mapping. The algorithm identifies all of the PAM sequences and extracts all possible corresponding spacers, on both strands of the sequences, from the sets of barcode pairs which were successfully mapped.

The successfully mapped gene barcode sets are translated into protein sequences. This produces a set of successful translations and poor translations. Poor translations contain “N” unknown bases or very short sequences due to premature stop codons or mutations. The successful translations and spacers are combined into a single data set.

Target sequences are used to filter this data set. Sequences in the target set become the set of target spacers, all others become non target spacers. The non-target spacers are combined with spacers from the plasmid vector, the poorly mapped sequences, and the poor translations to form a master set of spacers to avoid.

Collisions, in which a spacer sequence can be linked to molecules in the target set and the non-target set, if any, are identified. Any collisions are removed from the target set. This can be done either with the full spacer length or just the PAM-adjacent seed sequence. The seed sequences are 12 nt or 16 nt in length depending on the required stringency.

Optionally, the Levenshtein distance between either the full spacers or the seed sequences of the target sequences are compared to non-target set sequences to avoid a close Levenshtein distance between target sequences and non-target sequences. The minimum Levenshtein distance is typically set to 2.

The resulting set of selected spacers nucleic acid sequences is normalized with a normalization algorithm as follows. The median number of reads is calculated for all barcodes for each target sequence of interest. The median of all of the calculated medians in the target sequence population is calculated to arrive at the median number of reads for the entire library.

For all barcodes, the distance between the barcodes number of reads and the median of medians for the library is calculated. For each sequence of interest, three (or other predetermined number) spacers that are closest to the median of medians for the library are selected.

The resulting selection of normalized spacer oligonucleotides results in the target molecules having a more uniform distribution.

sgRNA Synthesis

Spacer sequences from barcode protospacers within DropSynth gene libraries are computationally selected as described above. Briefly as described below and as depicted in FIG. 11 using the sequences of Table 27 below, orthogonal primers, a T7 promoter for in vitro transcription, a scaffold overhang and a BsaI restriction site for golden gate assembly is added to each of the spacer sequences to generate spacer oligonucleotides. All spacer oligos are synthesized as a single OLS pool. Libraries are sub pooled from the OLS pool via PCR and golden gate assembly is performed to join the spacer oligos to a conserved, duplexed sgRNA scaffold sequence. The sgRNA template is added to a T7 in vitro transcription mix to generate the sgRNA pools. These are verified via TBE-urea gel electrophoresis and RNA-seq.

TABLE 27

| Sequence name | Sequence | Description | SEQ ID |
|---|---|---|---|
| ds_sgRNA_scaffold_ | GAGAACGGTCTCCTAGAAATAG | Duplexed sgRNA scaffold | 38 |
| BsaI_YG | CAAGTTAAAATAAGGCTAGTCC | DNA sequence from IDT | |
| | GTTATCAACTTGAAAAAGTGGC | | |
| | ACCGAGTCGGTGCTTTT | | |
| skpp15-6-F | CGCAGGGTCCAGAGT | Forward PCR primer Lib 15 | 39 |
| | | (DHFR-pEVBC8 barcode | |
| | | spacers) | |
| skpp15-6-R | GTTCGCGCGAAGGAA | Reverse PCR primer for | 40 |
| | | Lib 15 (DHFR-pEVBC8 | |
| | | barcode spacers) | |
| S. pyogenes NEB | mC*mA*mU* rCrCrU rCrGrG | Control sgRNA | 41 |
| control sgRNA | rCrArC rCrGrU rCrArC rCrCrG | | |
| | rUrUrU rUrArG rArGrC rUrArG | | |
| | rArArA rUrArG rCrArA rGrUrU | | |
| | rArArA rArUrA rArGrG rCrUrA | | |
| | rGrUrC rCrGrU rUrArU rCrArA | | |
| | rCrUrU rGrArA rArArA rGrUrG | | |
| | rGrCrA rCrCrG rArGrU rCrGrG | | |
| | rUrGrC mU*mU*mU* rU | | |

Sub Pool sgRNA Spacer Oligo Libraries from an OLS Pool

The OLS chip pool is resuspended and diluted according to the manufacturer's specifications. Briefly, the OLS chip pool is resuspended in nuclease free Tris-EDTA (TE) buffer, pH 8.0 to a concentration of at least 10 ng/uL. The stock OLS chip pool is diluted 1:10 and 10 μM of the forward and reverse sub pool amplification primers are prepared for each sub pool.

A qPCR reaction is run for each sub-pool to determine the number of cycles required for amplification. Amplifications are stopped several cycles before plateauing to prevent overamplification of the libraries. Alternatively, qPCR can be run for 30 cycles solely for determination of cycles required and the amplification product can be discarded.

qPCR reaction mixtures are prepared in duplicate for each sub pool and corresponding primer pair as indicated in Table 28 below and the qPCR reactions are run according to the protocol in Table 29 below.

TABLE 28

| | 1X reaction | |
|---|---|---|
| Component | (25 μL final vol) | |
| Nuclease-free water to fill | 7.5 | μL |
| KAPA HiFi HotStart ReadyMix (2X) | 12.5 | μL |
| Fwd primer (10 uM) | 1.25 | μL |
| skpp15-6-F | | |
| Rev primer (10 uM) | 1.25 | μL |
| skpp15-6-R | | |
| Chip9 OLS 1:10 (0.43 ng/ul) | 2.5 μL | (1 ng) |
| Thiazole green (100X) | 0.25 | μL |

TABLE 29

| Cycles | Step | Temperature | Time |
|---|---|---|---|
| 1 | Denaturation | 98° C. | 30 s |
| 40 | Denaturation | 98° C. | 10 s |
| | Annealing | 50° C. (for skpp15-6 | 15 s |
| | | primers) | |
| | Extension | 72° C. | 15 s |

Using the OLS pool as the template, the sub-pools are bulk amplified in the reaction mixture described in Table 30 below.

TABLE 30

| | 1X reaction |
|---|---|
| Component | (25 μL final vol) |
| Water to fill | 9.75 μL |
| KAPA HiFi HotStart ReadyMix (2X) | 12.5 μL |
| Fwd primer (10 uM) skpp15-6-F | 1.25 μL |
| Rev primer (10 uM) Skpp15-6-R | 1.25 μL |
| Chip9 OLS 1:10 (0.43 ng/μL) | 2.5 μL (1 ng) |

The PCR protocol is run as indicated in Table 31 below.

TABLE 31

| Cycles | Step | Temperature | Time | |
|---|---|---|---|---|
| 1 | Denaturation | 98° C. | 30 | s |
| 15 | Denaturation | 98° C. | 10 | s |
| | Annealing | 50° C. | 15 | s |
| | Extension | 72° C. | 15 | s |
| 1 | Final extension | 72° C. | 1 | min |

| | | |
|---|---|---|
| Hold | 10° C. | infinite |

The PCR products are column-cleaned using Monarch DNA clean up kit (NEB Cat: T1030). Five times the volume of binding buffer as the volume of the pooled PCRs is used to elute each sub pool using one 5 μg DNA clean-up column with 10 μL hot elution buffer. The PCR products are run on gel to identify higher molecular weight products indicative of over amplification or excessive low MW products indicative of chip synthesis issues.

The PCR products may be sized selected using gel extraction and the amount of PCR product quantified using 1×hs dsDNA Qubit kit.

Bulk Amplification of sgRNA Spacer Oligo Sub Pools

Sub pools are bulk amplified to obtain a sufficient quantity of sub pooled DNA for downstream golden gate assembly with scaffold sgRNA sequence.

For each sub pool, a qPCR is run to determine the number of cycles required for bulk amplification. Amplifications are stopped several cycles before plateauing to prevent overamplification of the libraries. Alternatively, qPCR can be run for 30 cycles solely for determination of cycles required and the amplification product can be discarded.

qPCR reactions are run in the reaction mixture described in Table 32 below and PCR conducted according to the protocol of Table 31 above.

TABLE 32

| | 1x reaction | |
|---|---|---|
| Component | (25 μL reactions) | |
| Nuclease-free water to fill | 9 | μL |
| Kapa HiFi Master Mix | 12.5 | μL |
| FWD primer (10 μM) | 1.25 | μL |
| REV primer (10 μM) | 1.25 | μL |
| Lib sub pool PCR product | 0.8 μL | |
| Add 10 ng total (S2 Lib 15) | (10 ng) | |
| Thiazole green (100X) | 0.25 | μL |

Each sub pool is bulk amplified by 8×PCR reactions per sub pool using each amplified sub-pool as the template for bulk amplification in reaction mixture described in Table 33 below.

TABLE 33

| | 1x reaction |
|---|---|
| Component | (25 μL reactions) |
| Water to fill | 9.25 μL |
| Kapa HiFi Master Mix | 12.5 μL |
| FWD primer (10 μM) | 1.25 μL |
| REV primer (10 μM) | 1.25 μL |
| Lib sub pool PCR product | 0.8 μL |
| Add 10 ng total (S2 Lib 15) | (10 ng) |

Each sub pool is run according to the PCR protocol described in Table 34 below for the number of cycles determined in the qPCR procedure above.

TABLE 34

| Cycles | Step | Temperature | Time | |
|---|---|---|---|---|
| 1 | Denaturation | 98° C. | 30 | s |
| 6 | Denaturation | 98° C. | 10 | s |
| | Annealing | 50° C. | 15 | s |
| | Extension | 72° C. | 15 | s |
| 1 | Final extension | 72° C. | 1 | min |

| | | |
|---|---|---|
| Hold | 10° C. | Infinite |

The PCR products are column-cleaned using Monarch DNA clean up kit, two times the volume of binding buffer as the volume of the pooled PCRs is used to elute each sub pool using one miniprep clean-up column (NEB Cat: (NEB Cat: T1017-2) with 30 μl hot elution buffer. The concentration of DNA is quantified using a Qubit kit.

Golden Gate Assembly (GGA) Preparation of sgRNA Spacer Oligo Sub Pools and Scaffold Oligo.

5× assemblies per sub pool were prepared using a 2:1 scaffold oligo: spacer oligo sub pool ratio. The scaffold duplex (100 μM) 1:100 by adding ul scaffold to 100 μl water, resulting at about 30 ng/μl. The spacer oligo sub pools are dot dialyzed to remove any residual salts that may reduce T4 ligase activity. A 50 μm membrane is placed on a 245 mm×245 mm dish (Polystyrene Corning) filled with DI water. The DNA sub pools are pipetted onto the membrane filter and dialyzed for 20 minutes. The DNA is pipetted into clean tube.

GGAs are prepared according to Table 35 below.

TABLE 35

| | S2 Lib 15 GGA 1x | |
|---|---|---|
| Component | (25 μL total volume) | |
| Duplexed scaffold oligo (83 bp) | 4 μL | (~111 ng) |
| Add 2 pmol/rxn, | | |
| Spacer oligo sub pool (97 bp) | 9.7 μL | (~63 ng) |
| Add 1 pmol/rxn | | |
| 10X T4 DNA ligase buffer (NEB B0202S) | 5 μL buffer mix | |
| Add 1:1 with supplemental 1 mM ATP | | |
| (NEB P0756) | | |
| T4 DNA ligase (NEB M0202) | 0.25 | μL |
| BsaI-HFv2 (NEB R3733) | 0.75 | μL |
| Nuclease-free water to fill | 5.3 | μL |

The GGA protocol is conducted as indicated in Table 36 below.

TABLE 36

| Step | Temp | Time |
|---|---|---|
| | 37° C. | 5 min |
| 100 cycles | 16° C. | 5 min |
| Heat inactivation | 80° C. | 20 min |
| | 12° C. | hold |

The GGA products are column-cleaned using Monarch DNA clean up kit, two times the volume of binding buffer as the volume of the pooled GGA products is used to elute each sub pool using one 5 μg DNA clean-up column with 20 μL hot elution buffer The concentration of DNA is quantified using a Qubit kit.

In Vitro sgRNA Synthesis

Using each GGA sub pool product as the template, in vitro transcription reaction per sub pool is prepared according to Table 37 below as well as a 0 pmol (no DNA) control.

TABLE 37

| | | | |
|---|---|---|---|
| | | 0 pmol | Lib 6 |
| Component | Volume | control | 1.48 pmol |

| | | | | | | |
|---|---|---|---|---|---|---|
| NEB Murine RNase | 0.5 | μL | 0.5 | μL | 0.5 | μL |
| inhibitor (NEB | | | | | | |
| M0314) | | | | | | |

| Nuclease-free water | To fill | 11.5 | ul | — |
|---|---|---|---|---|
| GGA products | Up to | — | | 11.5 μL |
| (full-length sgRNA | 11.5 μL | | | (148 ng, |

| oligos)~180 bp | | | | | 1.48 pmol) | |
|---|---|---|---|---|---|---|
| 2-3 pmol if possible | | | | | | |
| DTT (100 mM) | 2 | μL | 2 | μL | 2 | μL |
| NEB 10x reaction mix | 2 | μl | 2 | μL | 2 | μL |
| (NEB B9012SVIAL) | | | | | | |
| Ribonucleotide mix | 2 | μL | 2 | μL | 2 | μL |
| NEB (NEB N0466L) | | | | | | |
| NEB T7 RNA | 2 | μL | 2 | μL | 2 | μL |
| polymerase (NEB | | | | | | |
| M0251L) | | | | | | |
| Total volume | 20 | μl | 20 | μL | 20 | μL |

The transcription reactions are mixed, spun down and incubate at 37 C for 2 hours. Each reaction is treated with DNase I (NEB M0303) in a reaction mixture according to Table 38 below. The reagents in Table 38 below are added to the samples while the samples are in a cooling rack on ice.

TABLE 38

| Component | Per 50 ul reaction | |
|---|---|---|
| Transcribed sgRNA | 20 ul reaction from 3 | |
| DNase I reaction buffer (10x) | 5 | μL |
| DNase I (RNase-free) | 0.5 μL (1 unit) | |
| Nuclease-free water | 24.5 | μL |

The reactions are mixed, spun down and incubate at 37 C for 10 minutes. The transcribed sgRNAs are column cleaned with Monarch RNA kit (NEB Cat: T2040), eluted with 25 μL nuclease-free water into RNase-free 1.5 mL tubes and RNA yield is quantified with Qubit HS RNA kit (ThermoFisher Cat: Q32852)

The sgRNAs are run a TBE-UREA (10%) denaturing gel (Bio-Rad 4566036) to confirm sgRNA size. The gel is post stained with SYBR gold nucleic acid stain and the size of pooled sgRNA are compared to the NEB sgRNA control sequence. The RNA-seq of sgRNA pools are RNA sequenced as described in Example 1 above to determine any sequence biases and/or mutated spacer sequences resulting in the following sequence metrics.

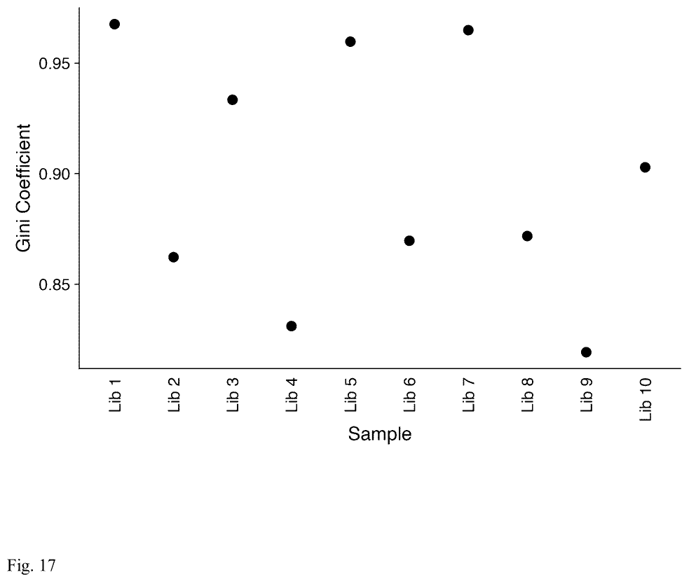

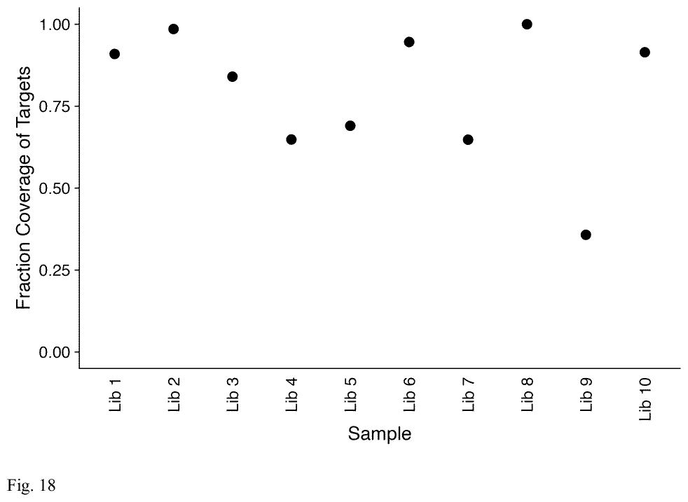

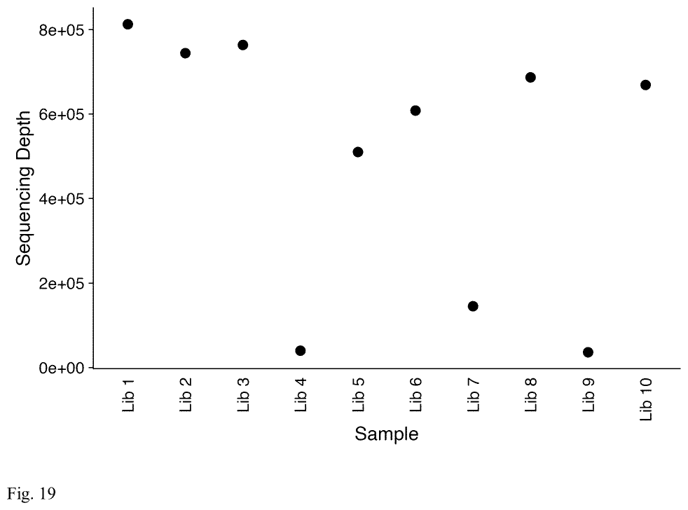

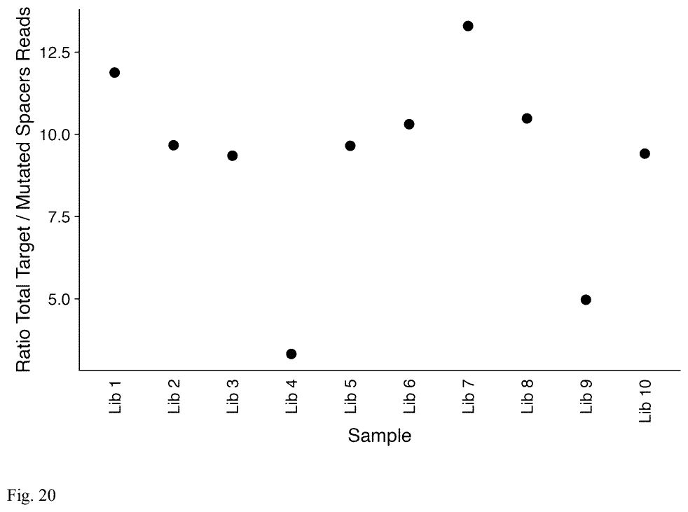

Lorenz curves are plotted for each library and the Gini coefficient for each library is determined. FIG. 17 plots the Gini coefficients (vertical axis) for each library (horizontal axis) demonstrating the variability of spacer oligonucleotide sequence distribution between libraries. FIG. 18 plots spacer sequence coverage (the fraction of all of the spacers in a library that were actually observed) on the vertical axis by library (horizontal axis). FIG. 19 is a graph that plots the sequencing depth (vertical axis) by library (horizontal axis) in the sequencing runs that resulted in the data of FIG. 17 and FIG. 18. FIG. 20 is a graph that plots the total number of spacer oligo sequence reads to mutated spacer reads (horizontal axis) by library.

### Example 6: Combined Targeted Enrichment Method

The sequences set forth in Table 39 below are used in the combined enrichment method of the invention.

TABLE 39

| Sequence name | Sequence | Description |
|---|---|---|
| Mi9_FWD_Amp_NV | AATGATACGGCGACCACC | DNA primer |
| | GAGATCTACACGCCGccga | |
| | acgaccgagcgcagcCATATG | |
| | (SEQ ID NO: 42) | |
| Mi9_REV_amp_NV | CAAGCAGAAGACGGCAT | DNA primer |
| (index 1) | ACGAGATTCGCCTTAcgaaa | |
| | agGAATTCtgcGacGtgACGT | |
| | Cgtg (SEQ ID NO: 43) | |

### Ribonucleoprotein Complex (RNPs) Preparation.

RNPs consist of the dCas9 protein complexed to single guide RNAs (sgRNA). sgRNAs provide targeted specificity once dCas9 recognizes a three-nucleotide proximal adjacent motif (PAM) on the DNA non-target strand. All dilutions and RNP preparation in this combined method are made in PCR tubes (0.2 mL) on a metal PCR cooling rack on ice.

sgRNA pools are diluted to 300 nM (˜11.3 ng/uL for sgRNAs that are 100 bases in length) with nuclease-free water. dCas9-3×FLAG-Biotin Protein (Sigma-Aldrich cat: DCAS9PROT-50UG) is diluted to 1 uM with dilution buffer following manufacturer instructions.

RNPs are prepared by adding reagents according to Table 40 below in the listed order.

TABLE 40

| | Enrichment | | No template control | |
|---|---|---|---|---|
| Condition | RNPs | | (NTC) RNPs | |
| RNP formation ratio | 3:1 | | 3:1 | |
| (dCas9:sgRNAs) | | | | |
| Component | | | | |
| Murine RNase inhibitor (NEB | 0.75 | μL | 0.75 | μL |
| cat: M0314S) | | | | |
| Nuclease-free water to fill | 19.25 | μL | 19.25 | μL |
| 10XNEBuffer r3.1 (NEB cat: | 3 | μL | 3 | μL |
| B6003S) | | | | |
| Pooled sgRNAs (300 nM) | 3 | μL | 3 | μL |
| Biotinylated dCas9 (1 μM) | 1 | μL | 1 | μL |
| Total vol: | 27 | μL | 27 | μL |

The reactions are mixed and pulse-spun in a microfuge. The samples are then pre-incubated at 25° C. in a thermocycler for 10 minutes and then incubated at 37° C. in a thermocycler for 10 minutes

### Enrichment Library Preparation

A total of 500 ng of library DNA is added to each enrichment reaction in volume of no more than 3 ul.

The enrichment reactions are prepared as described in Table 41 below.

TABLE 41

| | | |
|---|---|---|
| | | No template |
| Condition | Enrichment | control (NTC) |

| RNPs as prepared above | 27 | μL | 27 | μL |
|---|---|---|---|---|
| EVBC3-RFP scape DNA | 2.1 | μL | N/A | |
| (238 ng/ul) | | | | |
| Nuclease-free water to fill | 0.9 | μL | 3 | μL |
| Final vol: | 30 | μL | 30 | μL |

The reactions are mixed and pulse-spun in a microfuge. Then incubated at 37° C. for 15 minutes in a thermocycler.

Biotinylated dCas9 Pull Down with Streptavidin-Coated Magnetic Beads (NEB Cat: S1420S).

dCas9 is bound to targeted DNA barcodes. Beads are cleaned according to the manufacturer's instructions. 2×B&W buffer (10 mM Tris-HCl, 1 mM EDTA·Na₂, pH 7.5, 2M NaCl) is diluted 2-fold with nanopure water to make 1×B&W.

5 μl of beads are prepared for each enrichment reaction (including NTC). The stock bottle of beads is vortexed for 30 seconds and the required volume of beads are added to a 1.5 mL tube. 1 mL of 1×B&W buffer is added to the beads and the tube is placed on magnetic rack for 1 minute and the supernatant discarded. The beads are resuspended in 1×B&W buffer, and these steps are repeated 3 to 4 times. After the final wash, the clean beads are resuspended in 2×B&W buffer at volume that is 2× the starting bead volume.

10 μL of cleaned beads are added to nuclease-free 1.5 mL tubes and each 30 ul enrichment reaction (including NTC) is added to beads in 1.5 mL tubes immediately following enrichment incubation to a total volume of ˜40 μl.

The beads (1700 rpm) are shaken in thermomixer at 37° C. for 30 min.

Streptavidin-Coated Magnetic Beads Bound to Biotinylated dCas9 Washes.

The enrichments reactions containing beads are transferred to fresh 5 mL tubes and are washed nine times with 2 mL 2×B&W buffer. After washing, the enrichment samples are in 50 μL nuclease-free water. NTC is resuspended in 20 μL nuclease-free water.

### Proteinase K (NEB Cat: P8107S) Treatment

50 μL of resuspended, washed beads (from step 4c) are transferred to fresh 1.5 mL tubes. 1 μL proteinase K to is added to the enrichment samples. The samples are mixed, and pulse spun in a microfuge. The samples are incubated for 10 min at room temperature (˜25° C.) in thermomixer with shaking (1700 rpm). The samples are placed on a magnetic rack for one minute to separate beads from supernatant. The supernatant is collected and placed in fresh 1.5 mL tubs. The supernatant is column cleaned using Monarch DNA clean-up kit. 100 μL of DNA binding buffer is added to each sample and enrichment products are eluted with 20 μL of hot elution buffer in one 5 μg DNA clean-up column per sample (NEB Cat: T1034-2).

### Quantitative PCR.

qPCRs reactions are prepared according to Table 43 below with Q5 2× master mix (NEB cat: M0492) and 100× thiazole green (Biotium cat: 40086).

TABLE 42

| | 1x reaction | |
|---|---|---|
| Component | (50 μL total volume) | |
| Water to fill | 17.5 | μL |
| Q5 2X master mix | 25 | μL |
| Mi9_FWD_Amp_NV (10 uM) | 2.50 | μL |
| Mi9_REV_amp_NV (index 1) (10 uM) | 2.50 | μL |
| Enrichments/NTC (from step 53) | 2 μL (add after | |
| | aliquoting master mix) | |
| Thiazole green (100X) | 0.5 | μL |

DNA template is added to 48 μL of master mix/rxn described in Table 42 above. The PCR reactions are run according to the parameters described int Table 43 below.

TABLE 43

| Cycles | Step | Temp | Time |
|---|---|---|---|
| 1 | Denaturation | 98° C. | 30 s |
| 60 | Denaturation | 98° C. | 10 s |
| | Annealing | 72° C. | 30 s |
| | Extension | 72° C. | 30 s |

### Bulk-Amplification

Enrichment products are bulk amplified using 7× master mixes per enrichment sample and 2×PCRs for the NTC. The master mix is described in Table 44 below.

TABLE 44

| | 1x reaction | |
|---|---|---|
| Component | (50 μL reactions) | |
| Water to fill | 18 | μL |
| Q5 2X master mix | 25 | μL |
| Mi9_FWD_Amp_NV (10 uM) | 2.50 | μL |
| Mi9_REV_amp_NV (index 1) (10 uM) | 2.50 | μL |
| Enrichments/NTC (from step 53) | 2 | μL |

50 μl of each master mix is transferred PCR tubes and run according to the PCR parameters in Table 45 below.

TABLE 45

| Cycles | Step | Temp | Time | |
|---|---|---|---|---|
| 1 | Denaturation | 98° C. | 30 | s |
| Use cycle numbers | Denaturation | 98° C. | 10 | s |
| determined in step 6 | | | | |
| | Annealing | 72° C. | 30 | s |
| | Extension | 72° C. | 30 | s |
| 1 | Final extension | 72° C. | 2 | min |

| | | | |
|---|---|---|---|
| 1 | hold | 12° C. | infinite |

PCR products are column cleaned as follows. Samples are pooled g buffer cleaned using DNA binding buffer at the equivalent of 2× the volume of the pooled sample. Each enrichment sample is cleaned using a Monarch miniprep DNA clean-up column (NEB Cat: T1017-2) and 30 μL of hot elution buffer. The NTC is eluted with a 5 μg DNA clean-up column and 8 μl of hot elution buffer.

### DNA PCR Product Size Selection

A 2% Agarose gel (TAE) is prepared, loaded with PCR products and run at 115 V for 1 hour. The gel is stained with either SYBR safe (APExBIO Cat: A8743) or SYBR gold (ThermoFisher Cat: S11494) and the correct PCR products are size selected by cutting out correctly sized DNA bands. with a razor. The size-selected DNA is cleaned using the Monarch gel extraction DNA clean-up kit. One miniprep column per sample is used and the DNA is eluted with a 30 μL hot elution buffer.

200 to 300 ng of the DNA is nanopore sequenced and enrichment products analyzed.

### Example 7: Enrichment with Enzymatic Degradation of Linear Molecules

Cyclized perfects are selected by degrading linear imperfects with the enzymepas λ exonuclease and RecJf (see, Balagurumoorthy et al., 2008 Anal. Biochem. 381:172-74 which is hereby incorporated in its entirety by reference). Perfect sequence enrichment is evaluated by comparing sequencing reads from PacBio Sequel II or Oxford Nanopore before and after selection.

### Example 8: Enrichment Via Amplification of Cyclized Perfect Assemblies

Circular perfects are amplified using replication cycle reaction (RCR) (see, Su′etsugu et al., 2017. Nucleic Acids Res. 45:11525-34 which is hereby incorporated in its entirety by reference).

Perfect sequence enrichment is evaluated by comparing sequencing reads from PacBio Sequel II or Oxford Nanopore before and after selection.

Other embodiments will be evident to those of skill in the art. It should be understood that the foregoing description is provided for clarity only and is merely exemplary. The spirit and scope of the present invention are not limited to the above examples but are encompassed by the following claims. All publications and patent applications cited above are incorporated by reference herein in their entirety for all purposes to the same extent as if each individual publication or patent application were specifically indicated to be so incorporated by reference.

What is claimed is:

1. A method of enriching a predetermined nucleic acid molecule in a starting set of nucleic acid sequences comprising the steps of:
providing a starting set of nucleic acid sequences, the starting set comprising a plurality of nucleic acid sequences each of which comprises a unique subsequence,
contacting the starting nucleic acid sequence set with a nucleic acid targeting system that specifically binds to the unique subsequence of the of predetermined nucleic acid molecule,
separating nucleic acid targeting system from the starting nucleic acid sequence set, and
releasing the predetermined nucleic acid molecule from the nucleic acid targeting system such that a second nucleic acid molecule set if formed in which the predetermined nucleic acid molecule is enriched as compared to the starting nucleic acid set.

2. The method of claim 1, wherein the predetermined nucleic acid molecule is a DNA molecule, the starting nucleic acid sequence set is a DNA sequence set and the nucleic acid targeting system is an RNA guided targeting system.

3. The method of claim 2, wherein the RNA guided targeting system is a Cas9, Cas12, Cas13a, Cas13b, Cas12f, Cascade-Cas3, prokaryotic argonautes (*Marinitoga piezophila* (MpAgo), *Thermotoga profunda* (TpAgo), or *Rhodobacter sphaeroides* (RsAgo)) system.

4. The method of claim 3, wherein the RNA guided system is a CRISPR Cas9 system comprising a Cas9 nuclease and wherein the sequences in the starting DNA sequence set comprises a protospacer adjacent motif.

5. The method of claim 4, wherein the Cas9 nuclease is deactivated.

6. The method of 5 where in the protospacer adjacent motif is 5′-NGG-3′.

7. The method of claim 6, wherein the plurality of nucleic acid sequence in the starting nucleic acid set comprises at least 10², 10³, 10⁴, 10⁵, 10⁶, 10⁷, or 10⁸ nucleic acid sequences to about 10, 20, 30, 40 or 50×10⁹ sequences each of which comprise a unique random sequence.

8. The method of 7, wherein the starting nucleic acid sequence set comprises a plurality of predetermined nucleic acid molecule each comprising a size, wherein the size of the predetermined nucleic acid molecule is at least 100, 200, 300, 400, 500, 1000, 2000, 3000, 4000 or 5000 to about 10⁶ nucleotides.

9. The method of claim 8, wherein plurality of the predetermined nucleic acid molecules comprises a plurality of sizes, wherein the plurality of sizes is in the range of 10 to 5000, 50 to 4000, 100 to 3000, 500 to 3000 or 500 to 2000 nucleotides.

10. The method of claim 9, wherein the starting nucleic acid set comprises at least 100, 150, 200, 300, 400 or 500 ng of DNA and the enrichment reaction is run in a final volume of about 30 uls.

11. The method of claim 10, wherein the second nucleic acid sequence set is treated with proteinase K prior to quantification.

12. The method of claim 11, wherein the enrichment reaction is run for about 15 minutes to no more than 30, 45 or 60 minutes.

13. The method of claim 12, wherein the second nuclei acid sequence set is washed at least 6, 7, 8, or 9 times with a total wash volume of at least 2, 3, 4, or 5 mls. 10.

14. The method of claim 13, wherein a quantity of the predetermined nucleic acid molecule in the second nucleic acid set is enriched by at least one or two orders of magnitude as compared to a quantity of the predetermined nucleic acid molecule in the starting nucleic acid set.

15. The method of claim 13, wherein at least 30, 40, 50, 60, 70, 80, or 90% of the nucleic sequences in the second nucleic acid molecule set are the plurality of predetermined sequences.

16. The method of claim 13, wherein at least 40%, 50%, 60% 70%, 80% or 90% of each predetermined sequence is perfect.

17. A method of preparing a library of single guide RNA molecules, comprising:
providing a plurality of double stranded DNA oligonucleotide molecules wherein each oligonucleotide molecule comprises a set of 2 orthogonal primer sequences, a T7 promoter, a spacer sequence, a scaffold overhang sequence, a type 2 restriction site and a stop codon,
providing a plurality of double stranded scaffold fragment sequences having a 5′ end,
incubating the plurality of oligonucleotide molecules and scaffold fragment sequences with a type II restriction enzyme and a ligase in the same reaction mixture, wherein the type 2 restriction enzyme creates a 5′ overhang on the spacer oligonucleotide and on the scaffold oligonucleotide wherein the 5′ overhang on the spacer oligonucleotide is complementary to the 5′ overhang on the scaffold oligonucleotide thereby providing a library of assembled single guide RNA DNA template molecules, and
transcribing the single guide RNA DNA template molecules into a plurality of single guide RNA molecules.

18. The method of claim 17, wherein the double stranded DNA oligonucleotide molecules are prepared from a single stranded DNA oligonucleotide template by primer extension.

19. The method of claim 17, wherein the double stranded DNA oligonucleotide molecules are prepared from a single stranded DNA oligonucleotide template by PCR amplification

20. The method of claim 17, wherein the spacer sequence comprises, 5 to 100, 10 to 90, 12 to 80, 15 to 70, 16 to 60, 17 to 50, 18 to 40, 19 to 30, 26 to 72, 19 to 21 or 20 nucleotides.

21. The method of claim 20, wherein the spacer sequence does not comprise protospacer adjacent motif or type 2 restriction site.

22. The method of claim 21, wherein the plurality of scaffold fragment sequences to the plurality of oligonucleotide sequences is provided at a ratio of 2 to 1.

23. The method of claim 18, wherein the spacer sequences of the plurality of oligonucleotide molecules target more than 2, 20, 25, 50, 60, 70, 80, 90, 100, 10, 10², 10³, 10⁴, 10⁵, 10⁶, 10⁸ to about 10⁹ different nucleic acid molecules.

24. A sgRNA library produced by the method of claim 20.


SEQUENCE LISTING (43 sequences)

SEQ ID NO: 1 - DNA, 83 bp, synthetic construct
  source 1..83: mol_type = other DNA
gagaacggtctcctagaaatagcaagttaaaataaggctagtccgttatcaacttgaaaaagtggcaccgagtcggtgctttt

SEQ ID NO: 2 - DNA, 83 bp, synthetic construct
  source 1..83: mol_type = other DNA
aaaagcaccgactcggtgccactttttcaagttgataacggactagccttattttaacttgctatttctaggagaccgttctc

SEQ ID NO: 3 - skipped

SEQ ID NO: 4 - DNA, 21 bp, synthetic construct
  source 1..21: mol_type = other DNA
ttctaatacgactcactatag

SEQ ID NO: 5 - DNA, 26 bp, synthetic construct
  source 1..26: mol_type = other DNA
ttttagagctagaggagaccgttctc

SEQ ID NO: 6 - DNA, 14 bp, synthetic construct
  source 1..14: mol_type = other DNA
gggtcacgcgtagg

SEQ ID NO: 7 - DNA, 14 bp, synthetic construct
  source 1..14: mol_type = other DNA
gtgtggctgggaac

SEQ ID NO: 8 - DNA, 97 bp, synthetic construct
  source 1..97: mol_type = other DNA
gggtcacgcgtaggattctaatacgactcactataggcgttcaatttagattagggttttagagctagaggagaccgttctcgtgtggctgcggaac

SEQ ID NO: 9 - DNA, 97 bp, synthetic construct
  source 1..97: mol_type = other DNA
gggtcacgcgtaggattctaatacgactcactataggctattgtttctaaaatgcgttttagagctagaggagaccgttctcgtgtggctgcggaac

SEQ ID NO: 10 - DNA, 97 bp, synthetic construct
  source 1..97: mol_type = other DNA
gggtcacgcgtaggattctaatacgactcactataggagtgagtttaatctgggcgttttagagctagaggagaccgttctcgtgtggctgcggaac

SEQ ID NO: 11 - DNA, 54 bp, synthetic construct
  source 1..54: mol_type = other DNA
aatggccctgttgttttagagctagaggagaccgttctcgtgtggctgcggaac

SEQ ID NO: 12 - DNA, 97 bp, synthetic construct
  source 1..97: mol_type = other DNA
gggtcacgcgtaggattctaatacgactcactataggaaacttagcgtaatataagttttagagctagaggagaccgttctcgtgtggctgcggaac

SEQ ID NO: 13 - DNA, 97 bp, synthetic construct
  source 1..97: mol_type = other DNA
gggtcacgcgtaggattctaatacgactcactataggttctgtgcttaacaatgagttttagagctagaggagaccgttctcgtgtggctgcggaac

SEQ ID NO: 14 - DNA, 97 bp, synthetic construct
  source 1..97: mol_type = other DNA
gggtcacgcgtaggattctaatacgactcactataggagagcgcctcattcatgtgttttagagctagaggagaccgttctcgtgtggctgcggaac

SEQ ID NO: 15 - DNA, 97 bp, synthetic construct
  source 1..97: mol_type = other DNA
gggtcacgcgtaggattctaatacgactcactataggtattcgtatatgtgcgaagttttagagctagaggagaccgttctcgtgtggctgcggaac

SEQ ID NO: 16 - DNA, 97 bp, synthetic construct
  source 1..97: mol_type = other DNA
gggtcacgcgtaggattctaatacgactcactataggacggtgtcgcatttggaagttttagagctagaggagaccgttctcgtgtggctgcggaac

SEQ ID NO: 17 - DNA, 97 bp, synthetic construct
  source 1..97: mol_type = other DNA
gggtcacgcgtaggattctaatacgactcactataggttcggatcatgtcaacttgttttagagctagaggagaccgttctcgtgtggctgcggaac

SEQ ID NO: 18 - DNA, 97 bp, synthetic construct
  source 1..97: mol_type = other DNA
gggtcacgcgtaggattctaatacgactcactataggatcttaatgagtattgatgttttagagctagaggagaccgttctcgtgtggctgcggaac

SEQ ID NO: 19 - DNA, 97 bp, synthetic construct
  source 1..97: mol_type = other DNA
gggtcacgcgtaggattctaatacgactcactataggtagatagacctttcaccggttttagagctagaggagaccgttctcgtgtggctgcggaac

SEQ ID NO: 20 - DNA, 97 bp, synthetic construct
  source 1..97: mol_type = other DNA
gggtcacgcgtaggattctaatacgactcactataggaagaggtttgcgatctgcgttttagagctagaggagaccgttctcgtgtggctgcggaac

SEQ ID NO: 21 - DNA, 99 bp, synthetic construct
  source 1..99: mol_type = other DNA
gggtcacgcgtaggattctaatacgactcactataggttatgaaaattgtatgtcgttttagagctagaggagaccgttctcgtgtarggctgcggaac

SEQ ID NO: 22 - DNA, 97 bp, synthetic construct
  source 1..97: mol_type = other DNA
gggtcacgcgtaggattctaatacgactcactataggatccgattacgacaaatagttttagagctagaggagaccgttctcgtgtggctgcggaac

SEQ ID NO: 23 - DNA, 97 bp, synthetic construct
  source 1..97: mol_type = other DNA
gggtcacgcgtaggattctaatacgactcactataggtctaataccgcactctctgttttagagctagaggagaccgttctcgtgtggctgcggaac

SEQ ID NO: 24 - DNA, 97 bp, synthetic construct
  source 1..97: mol_type = other DNA
gggtcacgcgtaggattctaatacgactcactataggaacgcgtcccgttcatcggttttagagctagaggagaccgttctcgtgtggctgcggaac

SEQ ID NO: 25 - DNA, 97 bp, synthetic construct
  source 1..97: mol_type = other DNA
gggtcacgcgtaggattctaatacgactcactataggtgactctgcttagcctatgttttagagctagaggagaccgttctcgtgtggctgcggaac

SEQ ID NO: 26 - DNA, 14 bp, synthetic construct
  source 1..14: mol_type = other DNA
gttccgcagccacc

SEQ ID NO: 27-28 - skipped (2 entries)

SEQ ID NO: 29 - DNA, 49 bp, synthetic construct
  source 1..49: mol_type = other DNA
agcatatatcccggtctggannnnnnnnnnnnnnnnnnaaaagcaccga

SEQ ID NO: 30 - DNA, 42 bp, synthetic construct
  source 1..42: mol_type = other DNA
  modified_base 40..42: mod_base = OTHER; note = riboguanosine
gctaatcattgcaagcagtggtatcaacgcagagtacatnnn

SEQ ID NO: 31 - DNA, 22 bp, synthetic construct
  source 1..22: mol_type = other DNA
cattgcaagcagtggtatcaac

SEQ ID NO: 32 - DNA, 20 bp, synthetic construct
  source 1..20: mol_type = other DNA
agcatatatcccggtctgga

SEQ ID NO: 33 - DNA, 42 bp, synthetic construct
  source 1..42: mol_type = other DNA
tccagaccgggatatatgcttatgcggtgtgaaataccgcac

SEQ ID NO: 34 - DNA, 40 bp, synthetic construct
  source 1..40: mol_type = other DNA
cactgcttgcaatgattagccggtatcattgcagcactgg

SEQ ID NO: 35 - DNA, 20 bp, synthetic construct
  source 1..20: mol_type = other DNA
agagttcttgaagtggtggc

SEQ ID NO: 36 - DNA, 20 bp, synthetic construct
  source 1..20: mol_type = other DNA
gtgtggaattgtgagcggat

SEQ ID NO: 37 - DNA, 710 bp, synthetic construct
  source 1..710: mol_type = other DNA
atgtttccaagggcgaggaggataacatggctatcattaaagagttcatgcgcttcaaagttcacatggagggttctgttaacggtcacgagttcgagatcgaaggcgaaggcgagggccgtccgtatgaaggcacccagaccgccaaactgaaagtgactaaaggcggcccgctgccttttgcgtgggacatcctgagcccgcaatttatgtacggttctaaagcgtatgttaaacacccagcggatatcccggactatctgaagctgtcttttccggaaggtttcaagtgggaacgcgtaatgaattttgaagatggtggtgtcgtgaccgtcactcaggactcctccctgcaggatggcgagttcatctataaagttaaactgcgtggtactaattttccatctgatggcccggtgatgcagaaaaagacgatgggttgggaggcgtctagcgaacgcatgtatccggaagatggtgcgctgaaaggcgaaattaaacagcgcctgaaactgaaagatggcggccattatgacgctgaagtgaaaaccacgtacaaagccaagaaacctgtgcagctgcctggcgcgtacaatgtgaatattaaactggacatcacctctcataatgaagattatacgatcgtagagcaatatgagcgcgcggagggtcgtcattctaccggtggcatggatgagctgtacaaataa

SEQ ID NO: 38 - DNA, 83 bp, synthetic construct; identical to SEQ ID NO: 1
  source 1..83: mol_type = other DNA

SEQ ID NO: 39 - DNA, 15 bp, synthetic construct
  source 1..15: mol_type = other DNA
cgcagggtccagagt

SEQ ID NO: 40 - DNA, 15 bp, synthetic construct
  source 1..15: mol_type = other DNA
gttcgcgcgaaggaa

SEQ ID NO: 41 - RNA, 100 nt, synthetic construct
  source 1..100: mol_type = other RNA
  modified_base 1: mod_base = OTHER; note = methyl cytosine
  modified_base 2: mod_base = OTHER; note = methyladenosine
  modified_base 3: mod_base = OTHER; note = methyl uridine
  modified_base 97..99: mod_base = OTHER; note = methyl uridine
catcctcggcaccgtcacccgttttagagctagaaatagcaagttaaaataaggctagtccgttatcaacttgaaaaagtggcaccgagtcggtgctttt

SEQ ID NO: 42 - DNA, 58 bp, synthetic construct
  source 1..58: mol_type = other DNA
aatgatacggcgaccaccgagatctacacgccgccgaacgaccgagcgcagccatatg

SEQ ID NO: 43 - DNA, 62 bp, synthetic construct
  source 1..62: mol_type = other DNA
caagcagaagacggcatacgagattcgccttacgaaaaggaattctgcgacgtgacgtcgtg